(19) United States
(12) Patent Application Publication
(10) Pub. No.: US 2019/0111298 A1
(43) Pub. Date: Apr. 18, 2019

(54) BELT PRE-TENSIONING AND POSITIONING SYSTEM FOR TRAINING A MUSCLE

(71) Applicant: Robert Tremaine Whalen, Los Altos, CA (US)

(72) Inventor: Robert Tremaine Whalen, Los Altos, CA (US)

(21) Appl. No.: 15/951,016

(22) Filed: Apr. 11, 2018

Related U.S. Application Data
(60) Provisional application No. 62/485,712, filed on Apr. 14, 2017.

(51) Int. Cl. A63B 21/008; A63B 21/00

(52) U.S. Cl. CPC A63B 21/0085; A61B 17/135; A63B 21/4025

(57) ABSTRACT

A belt for use in a BFR system with an outer belt material hermetically sealed to an inner belt material along a perimeter, thereby forming at least one inflatable chamber, the inflatable chamber having an input port for accepting a gas into the chamber, the belt further comprising a first fastening means in communication with the outer belt material, for attaching to a second fastening means in communication with the outer belt material, thereby locking a circumference of the belt, when wrapped around a user's limb, and the belt further comprising a pre-tensioning system for prescribing and applying a known amount of initial compression to the limb by the belt.

### CROSS-REFERENCES TO RELATED APPLICATIONS

This application references in its entirety the previously utility patent applications Ser. No. 15/430,404, filed on Feb. 10, 2017, titled Barrel Belt by Whalen, and Ser. No. 15/428,141, filed on Feb. 8, 2017, titled Blood Flow Restriction Belts and System by Whalen, as well as claims reference and priority to previously filed provisional patent application 62/485712, titled Belt Pre-Tensioning System to Whalen, filed on Apr. 11, 2017 which are hereby incorporated by reference. The referenced patents herein described also reference additional patents and these other references shall similarly be incorporated herein by reference.

### FEDERALLY SPONSORED RESEARCH

Not applicable

### SEQUENCE USING OR PROGRAM

Not applicable

### BACKGROUND OF THE INVENTION

### Field of the Invention

This invention relates to blood flow restriction systems, and more specifically to a pre-tensioning and positioning system for a belt design for use therein, to provide a simple to manufacture, simple to use, comfortable, effective, and less expensive alternative to current designs and products in use.

### Background of the Invention

The muscle training apparatus, system, and method described in prior art, and herein in this application is spreading fast globally because of its beneficial effects as described below. In addition, national and foreign physicians as well as universities have conducted blood flow restriction research investigations, as a result of them, researchers have published many articles.

The muscle strength increasing method according to these patents is a distinctive non-conventional one that involves compression of an arm or leg at a position near the top thereof. This muscle strength increasing method (the subject muscle strength increasing method) is herein referred to as a “Blood flow restriction muscle training method” or simply BFR.

Muscles are composed of slow-twitch muscle fibers and fast-twitch muscle fibers. Slow-twitch muscle fibers are limited in their potential for growth. Accordingly, it is necessary to recruit fast-twitch muscle fibers in the muscle in order to develop the muscles. Recruitment of fast-twitch muscle fibers causes lactic acid buildup in the muscles, which triggers secretion of growth hormone from the pituitary. The growth hormone has effects of, for example, promoting muscle growth and shedding body fat. This means that recruitment and exhaustion of fast-twitch muscle fibers results in development of fast-twitch muscle fibers and, in turn, the entire muscle.

Slow-twitch muscle fibers and fast-twitch muscle fibers are different from each other in terms of the following. Slow-twitch muscle fibers use oxygen for energy and are recruited for low-intensity endurance activities. Fast-twitch muscle fibers provide for activities regardless of whether or not oxygen is present They are recruited after the slow-twitch muscle fibers for highly intense activities. Therefore, it is necessary to cause the earlier recruited and activated slow-twitch muscle fibers to be exhausted soon in order to recruit fast-twitch muscle fibers.

Conventional muscle strength increasing methods use heavy load with, for example, a barbell to cause the slow-twitch muscle fibers to be exhausted first, and then to recruit the fast-twitch muscle fibers. This recruitment of fast-twitch muscle fibers requires a significant amount of force generation from the muscle, is time-consuming, and tends to increase the burden on muscles and joints.

On the other hand, muscle exercise may be performed under the restriction of muscle blood flow into the limb distal to a predetermined position by means of applying pressure upon the muscles at the predetermined position near the top of the limb. Since less oxygen is supplied to these muscles, the slow-twitch muscle fibers, which require oxygen for energy, are thus exhausted in a short period of time. Muscle exercises with blood-flow restriction by application of pressure will result in recruitment of the fast-twitch muscle fibers without needing a large amount of exercises. More specifically, when pressure is applied circumferentially upon a limb at a predetermined position near the top of the limb, venous circulation is restricted while arterial circulation is kept almost the same as the normal condition if an appropriate pressure is applied. This is because veins are closer to the skin surface of the limb, and are thinner and less muscular (less resistant against an force for pressurization) than arteries while arteries are found deep within the limb, and are thicker and more muscular than veins. Venous pressure is also much less than arterial pressure. By holding that condition for a certain period of time, the limb that has compressed near the top thereof becomes engorged with blood which runs from arteries but cannot flow through veins. This promotes a state of blood pooling in the capillaries where such an amount of blood is not flowing normally. The limb that is compressed at a position near the top thereof gets into a state as if it were doing heavy exercises. During this time, because of the temporal occlusion of the veins, the muscle fatigue is caused by the fact that the lactic acid that has built up in the muscles is less likely to be removed from the muscles. Furthermore, the brain receives information of strenuous exercise from muscles, and brain's physiological action is then responsible for the production of much more growth hormone than is usually produced during the daily life for muscle regeneration as well as during typical exercises.

In other words, BFR training contributes to artificially produce a state which otherwise will occur during and after heavy exercises, it is possible to cause muscle fatigue much more heavily than would be produced normally with that amount of exercises. In addition, the user can “trick” the brain into secreting a larger amount of growth hormone.

Because of the aforementioned mechanism, restriction of muscle blood flow can allow users to significantly develop their muscles.

BFR training method is premised on the theoretical concept of the muscle strength increase by the restriction of blood flow. More specifically, the BFR training method involves the application of an appropriate force for pressurization to at least one of the limbs at a predetermined position near the top thereof to restrict the blood flow restriction into the limb distal to that position. The force for pressurization serves to put an appropriate stress attributed to blood flow decrease on the muscles. Thus, the muscles can be developed in an effective manner.

The BFR training method may feature a specific modality for muscle development without any exercises because it involves developing muscles by putting a stress attributed to blood flow decrease on the muscles. With this feature, the BFR training method is highly effective for the recovery of motor ability in people with impaired motor function, e.g., the elders or an injured person.

In addition, the BFR training method can compensate for a total amount of stress that is placed on the muscles by putting on the muscles a stress attributed to blood flow decrease. When combined with some exercises, the method advantageously reduces an exercise-related load as compared with conventional methods. This feature produces effects of reducing possible risks of joint- or muscle-damages and shortening a necessary time period for training, because it can decrease the amount of muscle exercises for the muscle development.

It should be noted that, for the implementation of the BFR training method, such a device or apparatus is essential that can restrict the blood flow through the muscles that are subject to be developed and that can precisely adjust and maintain the degree of blood flow restriction. A large part of this restriction effect, comfort, and safety in the case of pneumatic systems is the initial tension and positioning of the belt. Quantifying, making similar in the case of bilateral application, and making consistent from one session to the next is a feature of the product and system that is all but ignored, or at best loosely described in prior applications. Even researchers studying BFR fail to properly quantify the effects of BFR in relation to this initial tension and rely ultimately on the inflation pressure as the variable to measure in the study.

The importance of initial tensioning and positioning, particularly in the case of pneumatic belt systems can be easily seen by looking at extreme examples. In the case a pneumatic belt, or cuff, such as a blood pressure cuff, is placed around a limb extremely loosely, for example with 2 in of space between uninflated cuff and the arm, and the cuff is then inflated to its maximum, the cuff may reach the elastic limit of the fabric before it even contacts the skin. A cuff in this manner may be inflated above 500 mmHg without ever contacting the skin as the fabric tension takes up this stress and none of the pressure is transmitted to the user's skin. Conversely, if such a cuff is tightened extremely tightly, full occlusion may occur, and therefore no air is even required at all to fully cut of the flow of blood. As proper BFR desires to only restrict and not shut off blood flow, it is clear that quantifying, and making repeatable the initial starting tension is a critical component to proper and effective application of this muscle building technique.

Positioning is similarly critical for BFR belts because a narrower belt is designed to cover as little muscle mass as possible. With wider cuffs, positioning is less critical because there are not a lot of options. BP cuffs generally cover most of the bicep muscle for example, whereas the applicant's designs are meant to reside in the narrow region below the deltoid and above the bicep on the arm. Even if a certain pre-tension could be set, if there was not an easy and reliable way to adjust position and orientation on the limb without modifying the pre-tension, the belt would be difficult to use or require constant adjustment which would wear out fastening means in the case of hook and loop fastener and reduce life expectancy.

While previously filed applications describe the concepts involved in BFR training, and do mention in the case of U.S. Pat. No. 8,992,397 to Sato “pre-determined tension”, there is no discussion of the effects of the pre-tension on the belt, how to achieve “pre-determined tension”, or how to quantify or measure the pre-tension, etc. The reader shall note that “pre-tension” in this specification refers to the tension on the belt and accompanying compression on the body segment prior to inflation. The reader shall also note the term “tension” as described herein, and unless otherwise specified, shall refer to the tensile force in the belt that is wrapped around the body segment. The reader shall understand the purpose of the pre-tension, or tensile force in the belt is that it causes a related amount of radially inward compression on the body segment, and the reader shall understand clearly that more tension in the belt means more compression on the body segment, whereas tension and compression are typically inversely related if acting on the same body. While the concept of pre-tension is mainly relevant for pneumatic belts, where a further “tensioning” step is taken via inflation, the same concepts and measurement schemes and inventions herein may also apply to the non-pneumatic belts described in the prior art.

To elaborate, the goal of a BFR is to achieve a level of compression on the limb sufficient to restrict blood flow a certain amount so as to be able to complete a pre-determined protocol of sets and repetitions of different exercises. Too much compression and the user is too uncomfortable to continue, or worse in a dangerous situation of full blood flow occlusion, and too little compression and the training method is ineffective. Experimental evidence has shown that there is no prescription as to the level of restriction that is optimal across all people, and thus tuning the pressures to the “correct” level for each user is somewhat of a trial and error process. The overall compression level is created by several factors: the pre-tension of the belt on the limb, the pressure applied to the bladder of the belt, the design of the belt, and the underlying anatomy.

Therefore, in order to achieve consistent results and optimally efficient training, it is important to quantify and make repeatable as many of these variables as possible. Many pneumatic systems make the bladder pressure level readily available, but none of them accurately and efficiently quantify the initial belt tension around the limb prior to inflation. The fabrication of a belt design is generally consistent so once a user has dialed in the pre-tension and the bladder tension that are best, it is only important that they be able to repeatedly and easily re-apply these conditions for subsequent trainings. The underlying limb anatomy however can vary depending on the clothing worn (which impacts circumference) and how hydrated the user is, whether they were exercising previously etc. If a pre-tension setting therefore is provided for a belt, depending on the spring constant, it can have a dramatically different impact on the effective compression on the limb based on the actual state of the user and what clothes they are wearing. Therefore it is best to optimize the pre-tension system to be as insensitive to underlying girth of the limb/clothing as possible. Additionally, users frequently employ BFR bi-laterally, meaning on two arms or two legs at once and as pressures are generally similar for both limbs, it is critical that the pre-tension values be able to be measured consistently on each side so that one side doesn't fatigue and fail substantially earlier in an exercise period than the other. Have a system that is insensitive to underlying girth also makes it convenient as a left arm and typically also be used on a right arm for example and the user does not have to track which is which.

A positioning system, as will be described by the applicant, is similarly important for creating a maximally functional BFR belt such as the ability to place the location and orientation on the limb in an adjustable manner. Sato describes the deficiency with his prior designs in U.S. Pat. No. 8,992,397 in the background where he discusses the tendency to rotate and position certain members in positions that might interfere with movement or chafe the body. The applicant has discovered that it is advantageous to create two steps for donning a belt, vs. a single step as used in all prior art to the applicant's knowledge. Prior art pneumatic cuff designs attempt to simultaneously place the belt in its final location as well as apply the final desired amount of pre-tension in the same action. This makes the belt very difficult to apply, especially by oneself because if a belt is not perfectly sized, there is typically excess overlap and it's very difficult to keep the loose end against the limb while pulling the other end substantially further around the limb circumferentially. The longer the belt, the more difficult it is to wrap a limb by oneself while trying to control the tension at the same time because to get the wrap around the limb, one must hold one end to the limb with one hand and then wrap the other end around the limb with the other hand. However, to move circumferentially around the limb, the limb actually blocks the arm so you must let go and reach from the other side. But, as soon as you let go, the tension is lost so it's a very difficult process. Anyone who has tried to wrap an ace bandage around a leg or arm will understand the difficulty. A perfectly sized belt approximately the limb circumference might solve this problem, but then many different belt sizes would be needed, which makes manufacturing and logistics difficult.

The applicant's invention of a pre-tensioning and positioning system herein solve this issue by securing the loose end against the limb in an initial tensioned position and preventing the loose end from moving on the limb, and thus allow usage of both hands for securing the overall starting tension in the open position. Creating this intermediate position, termed the “initial tensioned position”, not only is unique to the applicant's inventions in that it makes initial tension adjustment simpler, but it also has three additional benefits.

First, it has been observed and mentioned in U.S. Pat. No. 8,992,397 to Sato that the belt is likely to rotate around the limb during placement and that this may be disadvantageous as it may position the input port, or a connector in a position that is hard to reach or that interferes with movement. It is therefore desirable to be able to adjust both the orientation on the limb as well as the location longitudinally along the limb when placing the belt on oneself. In the case of Sato's designs, or any belt that does not incorporate an intermediate “initial tensioned position”, it is not possible to adjust the belt once secured to the limb because the tension is too much and the friction against the limb surface is too great. This is especially true in the case a high friction surface is used to prevent slippage on the limb during usage. The location longitudinally along the limb is critical so as not to cover a nerve, and not cover major muscle groups like the biceps on the arm. The rotational location is similarly critical so as to position the input port correctly and keep any fasteners or abrasive edging away from the chest area. No cuff in the prior art that the applicant could find addresses creating this intermediate “adjustable” state as the applicant will later describe and then subsequently locking down the position on the limb. Sato's designs, or others could be adjusted rotationally and/or longitudinally on the limb if they were put on loose, but then the fastening means would need to be detached so that proper tension could be applied, thus negating the point of having set the belt tension loose enough to rotate the system in the first place. No prior art to the applicant's knowledge therefore allows this two-step process.

Secondly, it can be observed with prior systems that if the loose end of the belt that is overlapped and not secured to the body of the belt in some way, it may easily slip out from under the belt body. This effect is exaggerated the narrower the belt, which is problematic as the preferred profile of a belt for BFR training is narrow because it mitigates against occlusion. Whereas traditional BP cuffs or tourniquets are wide, and thus their inflated cross-sectional profile, or section modulus, is high and naturally resists lateral movement, the applicant's designs are narrow and risk the bottom layer slipping out from under the top layer if the loose end is not secured. The applicant's pre-tension solution simultaneously solves this problem as well as will be described later.

Finally, in the case a belt shrinks when inflated, and this shrinkage is part of what gives compliance and overall compression to the underlying muscles, it is important that the point of connection of the loose, overlapped end be fixed to the belt body to form the minimal circumference. As the belt shrinks, the overall tension in the belt increases, whereas if the loose end were not fixed to the bottom of the belt, it would just shrink with nothing to pull on and the belt would not be able to develop the same level of compression, nor would it be able to expand the same amount. As prior art belts are not designed to shrink in length, this is a new issue and inventive solution discovered by the applicant during testing of this invention.

The applicant will therefore disclose several concepts aimed at creating a simple, robust, and easy to use pre-tensioning and positioning system that allows for similar, consistent application of pre-tension to the belt prior to inflation. The application will further describe how these designs may be made prescriptive versus other systems that require trial and error every time in order to ascertain pre-tension values. The applicant will further explain how the applicant's invention is less prone to measurement error or conditions of use than other previously described methods. For example, if a simple marking system is suggested as in U.S. Pat. No. 6,149,618 to Sato, if a user is bare skinned or using a shirt underneath the effective circumference may be significantly altered so as to drastically change the effect of tension the belt to an “appropriate pre-determined tension” as prescribed by a marking on the belt. This has to do with the characteristics of the belt, where a stiff belt, while potentially having an elastic property, must be significantly stiff to restrict enough flow, but this necessarily means that it is extremely sensitive to the underlying circumference of the limb, which Sato's belts are.

The reader shall note that the applicant uses the term “circumference” throughout this application to describe the distance around an object, even if the object is not perfectly circular. For example an oblong shaped limb has a perimeter around a cross section, and the applicant refers to the distance of this perimeter as the body segment “circumference”.

When the tightening tool, or hereinafter referred to as “belt”, “band”, “belt”, “inflatable band” etc., is placed on the body, a certain amount of tissue is compressed inward (minimal amounts of tissue squish out the sides). Superficial veins are temporarily partially or fully occluded, depending on the level of compression, and tissue is pushed radially inward. Tissue is incompressible relative to the capillary, venous, and arterial systems, and thus, the compliance in the system is primarily the partial or total collapse of these vascular compartments, and secondarily the shift of extracellular fluid away from the zone of pressure.

This explanation is neglected in previous patent applications, leading one to surmise that the inventors did not fully understand what was happening inside the targeted limb regarding tissue displacement and fluid shifts. The requirements for occlusive cuffs are similarly more stringent in that zero leak or pass-through of blood is allowable whereas some amount of leakage is actually desirably with BFR. As will be disclosed, what is essential in the end then, is only to cause enough displacement in the correct areas on the human body, i.e. where veins are present superficially or in the deep system, as to achieve the required level of BFR. Inventors McEwen and Sato similarly fail to properly disclose simple, repeatable, cost effective solutions to quantify and make repeatable the application of the belt system with adequate initial pre-tension. Sato and McEwen describe in their applications the importance of reducing the cost of the system (Sato U.S. Pat. No. 8,992,397), improving the comfort level (McEwen U.S. Pat. No. 6,746,470), and making the system easy and safe to use, together with an instructor or by oneself (Sato U.S. Pat. No. 8,992,397), as the principal barriers to mass adoption, and it is the aim of the applicant to solve these deficiencies in existing products and disclosed embodiments.

U.S. Pat. No. 6,149,618

To the best of the applicant's knowledge, U.S. Pat. No. 6,149,618 to Sato is the original application on the subject of BFR and describes a simple, non-inflatable band concept and generic method of using the band to perform BFR training, or KAATSU Training™ method. Sato describes a simple band, or rope, made of elastic material, for wrapping around the body as a tightening tool, as well as a band or belt made of inelastic material with spring inserted and means to tell what tension is applied. It appears, Sato had not yet conceived of the method of using air pressure when coming up with this concept and thus there are no features in the design related to making an air-bladder based system function comfortably, effectively, and cheap to make. Sato correctly notes the importance of the tightening tool having some elastic property and some method of knowing what the compression force is that is applied to the body. While Sato discloses a liner for the belt, Sato only recognizes the importance to protect the user's skin from abrasion Sato, in U.S. Pat. No. 8,992,397, notes the economical nature of a non-pneumatic construction, and the importance thereof, however Sato's following patents are all utilizing a pneumatic adjustment, as is the KAATSU equipment currently on the market, and thus Sato has acknowledged the superiority of such a system from an adjustability, precision, safety, and efficacy standpoint. This is likely because such a construction, as described in U.S. Pat. No. 6,149,618, would be cumbersome for a user to try to adjust. It is tougher and more tedious to make small adjustments in the locked circumference compared to first locking a circumference and then adding small amounts of air pressure. Further, differences in friction along the surface of the limb lead to uneven tensioning and tightening with a non-pneumatic system as pointed out by McEwen in U.S. Pat. No. 6,746,470. Sato, correctly recognize the superiority of a pneumatic system.

Further, Sato does not address the need to provide a means for users to rapidly tension the belt in a simple and cost effective way. Using a scale as depicted is imprecise because the spring constants have to be relatively high in order to provide enough compression on the muscles. The flip side of this is that small inaccuracies in measurement from the user result in large differences in compression and may lead to unsafe conditions. Additionally, Sato's preferred fastening method is the hook and loop fastener, so just trying to apply the tightening tool initially will be cumbersome as the hook and loop fastener will tend to catch and make precise placement of the hook and loop fastener very difficult. This problem is further exacerbated by the situation when the hook and loop fastener must be connected behind one's arm because the user can't see that location and is “feeling in the dark”.

Sato further mentions having markings or a scale of where to place the belt, but this also must be done on the limb in order to be able to apply sufficient tension and therefore if the indicator has markings, the markings may be out of view of the user. This is in contrast to the applicant's invention where pre-tension levels may be set prior to application to the limb and are insensitive to chambers in circumference of the limb by wearing a shirt for example.

Sato's device is therefore meant for tensioning ON the limb, whereas the applicant's invention be me done prior to applying to limb in a defined, measurable, and non-removable setting so the user does it once and never has to think about it again.

Sato talks about the downside of pull back and overlap style belts and tendency to rotate around the limb and make tensioning difficult. If the belt itself is elastic as Sato prefers in all his patents, this means that significant initial tension must be applied in order to provide enough restriction, and rotation from applying this tension around the limb is problematic in addition to the frictional issues previously discussed. This is in contrast to the applicant's preferred design of an inelastic outer layer similar to the design of a standard blood pressure cuff. Inelastic pneumatic belts, especially when combined with the applicants referenced patent inventions, mean that strong initial tension is not necessary, whereas with KAATSU equipment it is. However, whereas a blood pressure cuff, which is non-elastic and pneumatic, is inflated until occlusion is detected by a technician, the applicant's design is meant for unsupervised use by the general public and therefore initial pre-tension measurement and prescription can greatly aid in making the system safe and effective for the general public.

The applicant's experiments and user feedback show that very fine adjustments to initial tension of a non-elastic belt, or belt with high spring constant such as KAATSU equipment, make a BIG difference in terms of the effect, and because Sato's spring in U.S. Pat. No. 6,149,618 is in-line with the main belt, it must be very stiff so as not to deform too much and provide enough compression during use. This necessarily makes it very sensitive and markings for reading so close to together it will be nearly impossible to make fine adjustments. While tensioning the belt on oneself, in the case where significant tension is required, the body must resist rotation and therefore muscles in the limb that have the belt tense and artificially increase the apparent belt tension. Slight body movements will cause muscle contractions which will move the needle on the dial and further make tensioning very difficult. While some trial and error could be undertaken and a marking left on the belt as Sato suggests, this takes time and patience, which most users don't have. Further, the belt is still extremely sensitive to clothing worn because of the nature of it being a stiff spring.

In the case of body movement when placing the belt on oneself, you actually don't know where the tension is until you fasten the belt down and then let go. In most cases this first guess is likely wrong, and so you adjust it, and do it again, and the process repeats with trial and error. Markings eliminate this but then are sensitive to underlying clothing, changes in limb girth, etc. because the belt which is being measured has a very stiff spring constant in order to apply enough compression. The applicant's design by contrast preferably has a low spring constant, or at least markedly lower than the outer layer of the belt itself when applied to the limb, and so changes in circumference have much less impact on final compression. A large circumference simply means the actual inflatable portion, when laid down will be a little bit bigger in circumference, but the initial pre-tension won't have dramatically increased and thus safety is maintained.

All in all, Sato's system for applying belt tension is imprecise and cumbersome from an ease of use standpoint. Sato himself in U.S. Pat. No. 7,413,527 says that his first invention is not so easy to do without someone's help and that this is a barrier to widespread adoption. This is evident further by the fact that Sato has completely moved away from such systems to pneumatic systems in commercialization.

U.S. Pat. No. 7,413,527

Sato then advanced to U.S. Pat. No. 7,413,527, in which he improved upon the simplistic band design with another belt design. Sato also mentions another deficiency of U.S. Pat. No. 6,149,618, which is that Sato's design in U.S. Pat. No. 6,149,618 rotates around the limb when a user attempts to tighten it by themselves. This is a problem with U.S. Pat. No. 6,149,618 because for that device to function, significant compression to the skin must be applied by the band at the outset, to achieve the required levels of compression. This rotation therefore renders the design very difficult to use by an individual, further limiting adoption as stated by Sato in later applications. However, Sato's solution in U.S. Pat. No. 7,413,527 is to add multiple additional belt members which further complicates the sewing process, adds materials, bulk, and cost to the system. In addition to frictional features, the applicant will disclose how high tensioning forces are not actually necessary for effective use.

Sato describes in U.S. Pat. No. 7,413,527 column 3, line 41, that the belt is placed in loop form with a loop larger in circumference than the underlying muscles. Sato therefore doesn't contemplate that the belt should be pre-tensioned prior to application on the limb which not only provides a desired initial compression, but also holds the orientation, and consequently has troubles with rotation on the limb that necessitate the extra complication in design, especially in the case of application to the arms.

Sato further does not contemplate the use of markings in this invention. Even if markings were provided, there can be a situation where for some limb sizes, the appropriate marking will be out of visibility of the user and not useful. Because Sato says that the loop must be BIGGER than the limb where the belt will go, it means the belt has no pre-tension feature prior to application onto the limb and so Sato does not contemplate the applicant's invention of a belt where the initial tension is set once, prior to applying to the limb and can, but not necessarily, remain in this position through multiple uses in order to apply consistent pre-tension each time.

Because Sato describes the belt as being elastic, the belt must have a high spring rate, or be quite stiff, and, as described above, will similarly be sensitive to clothing or changes in limb girth.

Most importantly, Sato doesn't contemplate a system that is prescriptive for example, based on a limb girth, as in the applicant's design.

Sato never addresses the need to determine the initial tension of the belt, and its corresponding initial compression of the limb, and further does not discuss the by-product of higher initial limb tissue compressions as a detriment to the comfort of Sato's designs. High initial tissue compression results always in some degree of blood flow restriction and with concomitant buildup of metabolites and lactic acid even at rest. Without sufficient venous return blood flow to remove these metabolic byproducts, the limb may become uncomfortable with a pain not easily tolerated by many people.

In failing to address the initial compression guidelines, or design elements, Sato further shows he has not thought to optimize the inflation scheme as it relates to comfort of the user. As described above, a tight band is uncomfortable. Furthermore, without a large air cushion separating the muscle from the outer stiff band of the KAATSU belt, when the muscle contracts during an exercise, the muscle comes in direct contact with the outer band. This puts a higher localized stress on the muscle and can lead to pain and cramping. The user may choose to discontinue BFR Training. The applicant has witnessed such effects first hand with current KAATSU equipment where this local or point compressive loading and rubbing create the sensation that the hip flexor muscle was hit with a hard object. It is an aim of the applicant to solve this problem by spacing the inflatable bladder off the limb by a sufficient amount that it may inflate inward to form a larger air cushion than that of Sato's designs, further assisted by a body interfacing component that spreads the load on the limb.

Overall, Sato fails to recognize that the construction of the band has unnecessary components in it, and that sufficient, radially inward, more comfortable, and easier to apply, compression, can be achieved with proper construction techniques and selection of materials as will be disclosed by the present inventor. By stating that economics are important, yet including unnecessary components in the main band construction in this application and in now-current product sales, Sato unnecessarily complicates the design, driving up manufacturing costs, and increasing the price to the end customer, and shows that he has not contemplated a simpler more efficient design like that disclosed by the application.

U.S. Pat. No. 7,455,630

Sato then moved to U.S. Pat. No. 7,455,630 wherein Sato depicts a simplified BFR system consisting of a manual analog valve readout, and manual squeeze ball inflation means. However, rather than expanding on a full system that would be cheap and effective to implement, Sato continues to invent around methods of directing the inflation toward the user's limb with complicated limiting plate designs.

Sato additionally fails to further describe the importance or methods around how proper pre-tension should be achieved, or how a intermediate “position” state can be created. Sato acknowledges that a preferred device can provide accurate control of the compression force, but says on column 3, line 33, that the pressure on the muscles is provided by air and makes no mention as to the effect and importance of the pre-tension force and how this factors into the overall pressure. Sato discusses the need to prevent air gaps between the belt and the limb in column 7, line 43, but goes no further and does not discuss or disclose any mechanism, system, or method for properly applying pre-tension to the belt or making the belt easy to position on the limb. Further, as Sato describes the belt being substantially similar to U.S. Pat. No. 7,413,527, but with a different stiffener mechanism it is clear that Sato has still not figured out the importance of pre-tension or how to provide the user with a cost-effective, repeatable solution. All in all, U.S. Pat. No. 7,455,639 to Sato has the same deficiencies as U.S. Pat. No. 7,413,527, and further reinforces that Sato never contemplated the simplifying elements of the applicant's invention.

U.S. Pat. No. 8,992,397

In U.S. Pat. No. 8,992,397 to Sato, Sato comes back to the band design as a critical element to improve and reiterates, and further reveals, significant shortcomings of his previous inventions. Sato recognizes the superiority of a pneumatic system in improving the safety and pressure adjustment capabilities during setup and in the middle of a training session, but acknowledges the complexity in the design as a detriment to a pneumatic system versus a simple elastic band. Sato fails to recognize a design that is both simple and inexpensive to construct, and incorporates, and improves, the benefits of using pneumatics to apply pressure to the user. Sato describes two band structures, a straight type, and an overlap type, and how overlap types have a significant drawback of rotating on the user's arm when trying to apply initial tension, a problem that the applicant's invention solves. Because of Sato's band design, and the lack of a means to stand the band off the skin surface, the initial tension of a substantial degree is required to provide enough starting compression to obtain a sufficient overall compression level on the limb. In addition to solving the rotational problem, the applicant's invention does not require strong initial tension to achieve the required limb compression for most individuals, and therefore eliminates the rotational issues, while maintaining a simple construction. Sato further describes an overlap type of having a detriment that the ring employed, may cover a muscle region during rotation and cause discomfort the user, thereby further acknowledging the critical nature of user comfort in the application. Because the applicant's design does not require significant initial tension the displacement of the ring is not a problem. Further, the applicant has invented a solution for an overlap style belt that resists rotation and is easy to put on by oneself, which is something Sato had not solved, thus leading to the invention of U.S. Pat. No. 8,992,397.

Regarding rotation, Sato's invention in U.S. Pat. No. 8,992,397 must be pulled with both hands but both hands may not be functional if one were injured. The applicant's design by contrast may be applied by a single arm and thus is more versatile. The pre-tension design of the applicant's invention may be done prior to application on the limb by one arm and then the tension required is small enough that no rotation is seen meaning a single arm is sufficient for applying the belt. Sato's belt also only has two states, completely undone and full tensioned on the limb so there is no intermediate state that allows for proper positioning, orientation, and pre-tensioning. This means Sato's design likely requires multiple attempts to get the tensioning right, particularly since, in the case of arms, the arm under restriction must be contracted to perform the tightening which changes the musculature underneath to unpredictable state. Further for a small arm, the belt wraps around quite a lot and is difficult to tension because the fastening means may be pulling parallel and in the same direction with both hands, thus not actually putting much tension on the belt, but more pulling the arm in one direction. If one end we grabbed closer to the intersection point it may be possible to tension, but then the fastening means is far away and not possible to fasten down. This further illustrates the lack of an intermediate state and the importance there for simple, consistent pre-tensioning.

Sato further neglects to expand beyond the term “tensioned appropriately” and because of the belt design being stiff as previously described, has the same shortcomings as before. Because Sato's loop, as depicted in Fig A is larger than the limb where the belt will be placed, Sato does not contemplate a belt that is pre-tensioned prior to placement on the limb. In column 6, line 25, Sato explicitly states that the length formed when making the loop to put over the arm is larger than the circumference of the site of muscles to be compressed, even with the belt being elastic, so Sato is clearly not contemplating a belt whose tension may be set before placement on the limb.

The need for a second tensioning belt also adds bulk to the system, which is a detriment to athletic training. The added bulk and width leads in practice to interference and chaffing with the body segments as experienced when using KAATSU belts designed based off this patent application.

Sato further in column 17, line 6 acknowledges that with this design the belts can actually be over-tensioned. This can be dangerous and painful in training. This is a stark contrast to the applicant's design where the pretension may be prescribed such that this is not the case and the tension of the non-stretch portion of the belt is similarly pre-determined and prescribed so that this case of danger and pain will not exist.

Sato's concept for an overlapping belt, and his requirement that the inflatable portion cover the entire range of muscles to be compressed, leads to a very long and bulky band. For smaller users, the band may wrap more than once around the limb and this creates an annoying bump on the limb that interferes with normal movement of the arm or leg. It further makes it difficult to tension the band as just described, so getting proper consistent setup with a small limb is not easy. This is a big problem as it alters natural movement and will prohibit use in areas like performance training and rehabilitation where proper form and movement are necessary. The applicant's previously filed application for a band that shrinks in circumference when inflated actually reduces its circumference and moves inward, and has a thinner profile (because of no limiter plates and other components), staying much tighter against the skin and ameliorating these interference problems.

KAATSU bands also come in 6 different sizes (ranging from 18-70 cm), however because of the applicant's discovery that the entire limb doesn't need to be compressed, in combination with the applicants overlapping fold-back design disclosed herein, the applicant's invention is able to accommodate a larger range with only 4 sizes of bands ranging from 19-77.5 cm. This reduction in parts means better economies of scale and easier manufacturing and inventory management.

KAATSU bands are further not meant to be washed and therefore are prone to accumulating bacteria and sweat. The applicant will disclose design features and systems for making maintenance of the belts simple, effective, and sanitary.

### US2014/0324097 A1

Application US2014/0324097 A1 to Sato discloses a non-pneumatic belt with pressure sensors against the skin and a tension device to activate and deactivate a display device. Sato's new design requires a pressure measurement device and sensors and electronics which increase cost and reduce reliability as now batteries are needed. Presumably Sato is trying to solve the uneven pressure solution that friction causes when wrapping a non-pneumatic belt tightly around the skin produces. But just having pressure sensors doesn't actually change the behavior of the belt, and there will still be this deficiency.

Further, as described above relating to U.S. Pat. No. 6,149,618, the act of tensioning the belt on oneself causes contraction of the underlying musculature to some degree and so actual readings can't be read until the tension strap is applied and released. This means in practice that appropriate initial tension can't be prescribed and is subject to the same frustrating trial and error procedure as Sato's invention in U.S. Pat. No. 6,149,618.

Sato acknowledges need for multiple sensor units under the belt in [0014], but this adds unnecessary cost to the product and again, there is no guarantee of even pressures because of the friction against the skin.

Sato describes the tension sensor for use only as an auto-turnoff power saving feature, not a limit or alert feature to the user, so Sato still isn't prescribing or telling users how to apply tension based on a limb size. And as stated above, were the reading used to gauge the initial tension, it would be a frustrating trial and error solution that would be highly sensitive to changes in the user's underlying clothing or limb size over time.

Sato talks about displaying an actual and a “proper” pressure but this is the same problem as his original patent, that in order to tension the belt, one ends up contracting arm muscles underneath the belt, so the reading is artificially high, and subsequent relaxing drops the actual pressure lower so it is impossible to do this method without trial and error. This will wear out the fastening member in case of hook and loop fastener. Sato recognizes that this repeating application/removal process will occur in [00765] where he builds in that a plurality of pulling operations, are factored into an algorithm and not counted by the processor. Further, [0085] explicitly points out that the pressure may not be as desired and so the user must re-adjust it. Finally, Sato's failure to recognize the need and provide a solution for a safe, repeatable, low cost, and simple to use pre-tension system is evidenced by the fact that the belt is not in loop form prior to applying to body and all the tensioning operations are performed will applied to the limb.

### KAATSU Equipment

While Sato does not disclose any method for pre-tension prescription and measurement, KAATSU devices teach a “base pressure” measurement technique as part of the donning procedure. This process consists of applying the belt to the limb, inflating the belt with an amount of air, and watching a pressure reading. It is unclear exactly what is happening inside the device that is automatically controlling the pressure. In practice, this still leads to a trial and error effort and has the same susceptibility to limb size changes (due to clothing or otherwise) and this method is not taught to be used with a marking scheme. In practice it is cumbersome and because the volume of air is so small and the belt is so stiff, it is very cumbersome to use. KAATSU, and Sato for that matter, do recognize the need for “appropriate pre-determined tension”, they just fail to provide a system that is simple to use, repeatable, low cost, and not susceptible to changes in underlying girth, and that allow for reposition of the belt easily on the limb.

U.S. Pat. No. 6,746,470

U.S. Pat. No. 6,746,470 to McEwen describes an emergency tourniquet device for shutting off blood flow by military personnel in the field. McEwen's invention cannot be pre-tensioned, it must be wrapped around the limb in the field. Additionally, McEwen is only concerned with shutting off blood flow and so having an idea of the pre-tension on the belt is not something that would be important to him whereas in the applicant's field of use, it is very important to have a consistent and reasonable degree of pre-tension in order to have effective and safe training.

This is further evidence that the initial tension of McEwen's design is pre-determined and fixed by the geometry of the system. McEwen has designed his system to be operable by a single limb and in doing so, the cam mechanism that McEwen activates when pulling on the belt is activated when a certain force is reached by pulling on the strap. This force is the same regardless of what the underlying limb size is and therefore will be the same for all users. Again, McEwen is only concerned with getting the cuff snug (he mentions 20 mmHg) against the limb. By not having any adjustability, and not mentioning anything about prescriptive pre-tension, McEwen has not contemplated this need. In the case of a tourniquet where the only goal is to shut off flow completely, this inaccuracy in imprecision is acceptable, but it is not suitable for use with BFR where fine adjustments are necessary.

In fact, the applicant's invention of a pre-tension and positioning system that can be set up while NOT on the limb, would mean that the loop needs to be stretched over an injured limb in case of McEwens intended application therefore another example of how the applicant's invention is very different from McEwen's.

Throughout all of Sato's iterations, there isn't a description that the applicant could find of the importance of pre-tension and its effect on the overall compression level, how to apply appropriate pre-tension, nor how to prescribe what pre-tension to apply, or how to create an adjustable state on the limb. As the applicant has been in commercial production with the applicant's referenced application designs, and the most common question from users is around how tight the belt must be to start, it is clear this is a major missing component of both the applicant's, Sato's and McEwen's designs. Whereas McEwen's goals in the cuff design are to occlude, and therefore initial tension is less critical, it is especially important that Sato has need come up with a solution to this problem.

The applicant has taken an integrative approach therefore to solve this issue of pre-tension with a belt design and method of application that has fewer components, is simpler to construct, and performs better in terms of comfort to the user, and provides simple prescriptive guidance to the user to avoid trial and error effort by the user as well as providing an intermediate adjustment state where the position and orientation of the belt can be dialed in. The applicant's design simultaneously solves other problems of the narrow inflatable belts in terms of securing the position of the overlapped portion laterally from slipping out from under the overlapping portion. The applicant's design leads to an optimal belt design and BFR training system over current inventions and existing products.

The applicant's prior designs named in referenced patents above have also be in commercialization, and a common complaint is that it is difficult to make both limbs “the same”. The applicant has observed in practice that pretension when the outer belt is stiff or non-stretch altogether is extremely sensitive and therefore a design in which the tension prescription comes only from the outer stiff portion of the belt will always be very difficult for the user to apply consistently and evenly in the case of bilateral application. The applicant herein aims to solve this pre-tension problem with an apparatus and method for accurately, repeatedly, and prescriptively applying pre-tension to a pneumatic belt prior to inflation.

### BACKGROUND OF THE INVENTION

### Objects and Advantages

Accordingly, besides the objects and advantages of a belt for use in a blood flow restriction system described in this specification, several objects and advantages of the present invention are:
a) to provide a pre-tensioning system that is prescriptive
b) to provide a pre-tensioning system that is repeatable from one session to the next
c) to provide a pre-tensioning system that can be set once and left intact so the user doesn't need to set the tension each time they use the belts
d) to provide a pre-tensioning system that is inexpensive
e) to provide a pre-tensioning system that is simple to use with one arm
f) to provide a pre-tensioning system that is flexible to apply either prior to placement on the limb or while placing on the limb.
g) to provide a pre-tensioning system that is insensitive to the small changes in the underlying circumference whether it be due to a limb girth change, the user wearing other clothing or no clothing, etc.
h) to provide a pre-tensioning system that doesn't require the applicant to read a readout while simultaneously trying to apply the belt to their limb
i) to provide a pre-tensioning system that allows the stiff or non-stretch outer layer of the belt to be applied with little to no tension, reducing the tendency to rotate on the limb.
j) to provide a pre-tensioning system and belt design that does not need to fully encircle the limb to provide adequate BFR.
k) to provide a pre-tensioning system that is not bulky
l) to provide a pre-tensioning system that starts smaller than the limb and may be stretched for placement over a pre-determined range of muscles at a pre-determined, prescribed tension.
m) to provide a ratio-metric pre-tensioning system that prescribes a pre-tension setting for the belt based off one or more parameters of the user such as limb girth, intensity level, etc.
n) to provide a pre-tensioning system that provides consistent and even bi-lateral levels of initial tension
o) to provide a pre-tensioning system that resists a loose end of the belt from slipping laterally relative to an overlapping portion of the belt body.
p) to provide a pre-tensioning system that resists a loose end of the belt from moving longitudinally relative to the belt body as the belt tries to shrink.
q) to provide an intermediate state where the belt may be adjusted rotationally and/or longitudinally on a limb in order to get pre-inflation position correct before locking the position and inflating.

Still further objects and advantages will become apparent from a consideration of the ensuing description and drawings.

### SUMMARY

In accordance with the present invention, a belt is provided for use in a blood flow restriction system, the belt comprising an outer belt material and an inner belt material, coupled together in such a manner as to create at least one chamber to be inflated with a gas, preferably air, and a pre-tensioning system coupled to the belt that provides for prescriptive settings which may be based on a parameter of the user, and allows an initial tension to be created around a limb prior to inflation in order to provide more consistent and even radial compression, and in the case of use on right and left sides, similar bilateral compression, in addition to securing the belt in place to allow for simple application of the outer circumference of the belt in preparation for inflation.

### DRAWINGS—FIGURES

FIG. 1A—shows a belt configured in an overlap style with fastening means and spring element attached at one end.

FIG. 1B—shows a belt configured in an overlap style with fastening means and spring element attached along the length of the belt, away from either end.

FIG. 1C—shows a belt configured in an overlap style with fastening means and spring element attached along the length and facing the same direction as the third fastening means.

FIG. 1D—shows a belt configured in an overlap style with fastening means and spring element attached along the length and the spring element configured in a fold back style with the first fastening means folding back to attach to the inner surface.

FIG. 1E—shows a belt configured in an overlap style with fastening means and spring element attached along the length and the spring element configured in a fold back style with the first fastening means folding back to attach to the outer surface.

FIG. 1F—shows a belt similar to 1A but with a single chamber vs. multiple chambers.

FIG. 1G—shows a belt similar to 1A but formed as an integrally formed component with locking means.

FIG. 1H—shows an belt where a portion of the belt body is stretchable and constitutes a spring element.

FIG. 2A—Shows a belt configured in an initial fitting position prior to placement around a body segment.

FIG. 2B—Shows a belt configured in an expanded fitting position prior to placement around a body segment.

FIG. 2C—Shows a belt configured in an initial tensioned position around a body segment.

FIG. 2D—Shows a belt configured in an open position around a body segment where the outer layer of the belt is of high spring constant or non-stretch.

FIG. 2E-1—Shows an end cross sectional view of a belt in closed position around a body segment where an inflatable bladder is filled with a pre-determined amount of air.

FIG. 2E-2—Shows a side cross sectional view of the configuration of FIG. 2E-1.

FIG. 2F-1—Shows an end cross sectional view of a belt in a working position around a body segment where an inflatable bladder is filled with a pre-determined amount of air and the underlying musculature has been contracted.

FIG. 2F-2—Shows a side cross sectional view of the configuration of FIG. 2F-1.

FIG. 2G—Shows a belt wrapped around a limb in an open position with non-linear edging deforming to create a soft interface with the body segment for improved comfort.

FIG. 2H—Shows a cross sectional diagram of how the inner circumference of the belt changes between the open, closed, and working positions to compress the underlying body segment to restrict blood flow, but expand when muscles are contract to reduce spikes in surface pressure on the body segment during muscle contractions.

FIG. 3—Shows a detachable body interfacing component added to the belt of FIG. 1A

FIG. 4A—Shows a flow chart of how a user related pre-tension can be predicted, prescribed, and set up prior to being applied to a limb to prepare a pneumatic belt for inflation.

FIG. 4B—Shows a flow chart of how a user related pre-tension can be predicted, prescribed, and set up while simultaneously being applied to a limb to prepare a pneumatic belt for inflation.

FIG. 5A—Shows a perspective view of a wrap around style pre-tension system in an initial position with a loop top surface for attaching a fastening means wherein the pre-tension loop is formed with a body interfacing component with optional friction surface and held in place with a lateral stabilizer.

FIG. 5B—Shows the wrap around style pre-tension system when it is engaged in an initial fitting position prior to expansion for placement on the limb.

FIG. 6—Shows a separate pretension system comprising a stretchable component that may be a body interfacing component with a fastening means to fixing an inflatable belt body along the surface at one or multiple positions wherein the stretchable component sets the pre-tension amount and anchors the inflatable belt for wrapping around the limb to prevent rotation as the inflatable belt is moved to the open position.

DRAWINGS -- REFERENCE NUMERALS

90 - body segment
100 - Belt
101 - inner face
102 - outer face
103 - inflatable chamber
104 - input port
110 - first fastening means
110a - body interfacing first fastening means
111 - second fastening means
111a - body interfacing second fastening means
112 - length
113 - spring length
114 - spring element
115 - third fastening means
116 - fourth fastening means
117 - second inflatable chamber
118 - movable inner wall
119 - movable outer wall
120 - height
121 - width
122 - cutout
123 - edge
124 - belt body
125 - markings
126 - inner belt material
127 - outer belt material
128 - coloring element
129 - chamber connection joint
130 - loop coupler
131 - printable section
132 - loose end
201 - loop
202 - belt inner circumference
203 - body segment circumference
204 - omitted
205 - enclosed area
301 - body interfacing component
302 - friction surface
400 - integrally formed component
401 - locking means
501 - lateral stabilizer

### DETAILED DESCRIPTION

### Preferred Embodiment—Description

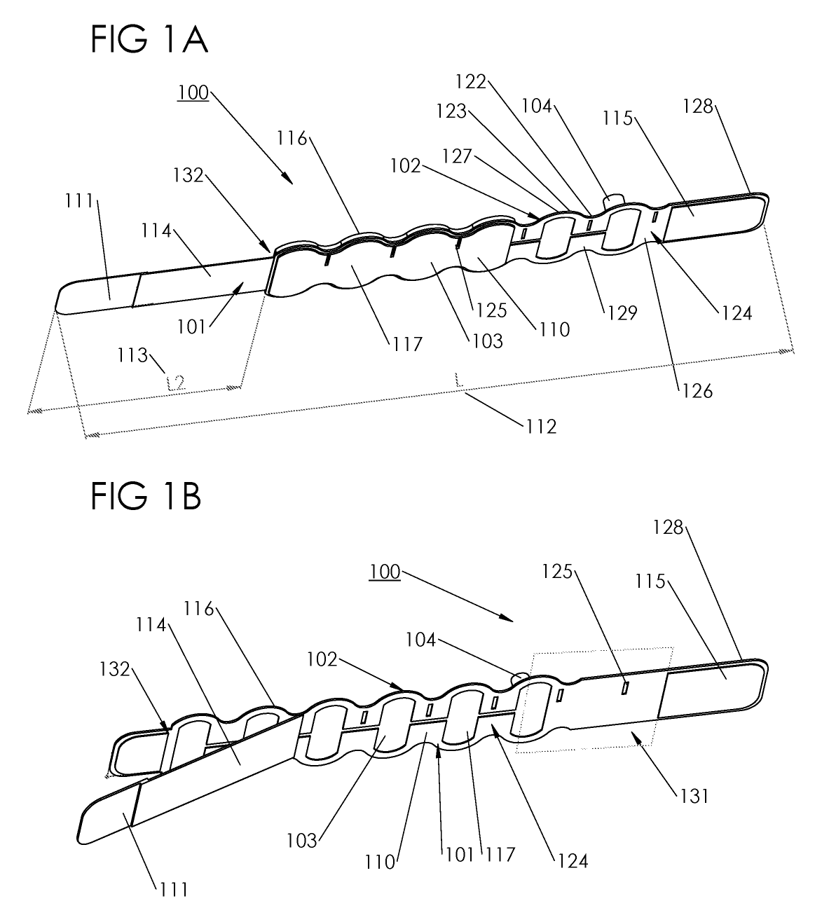

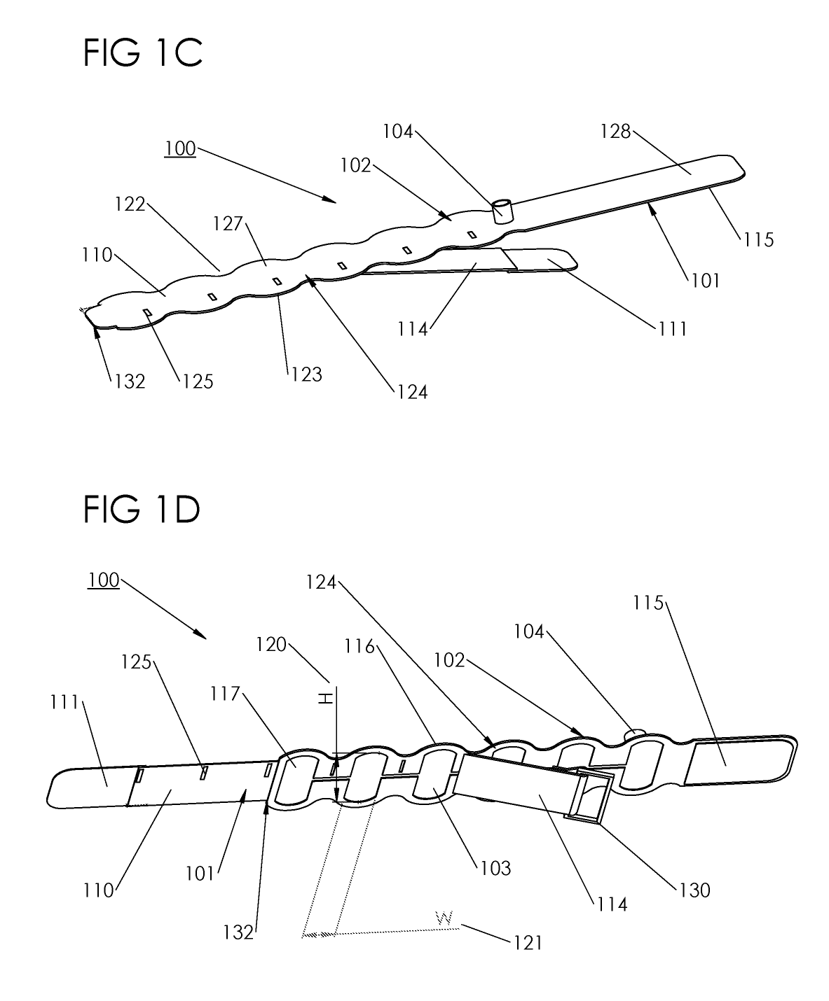

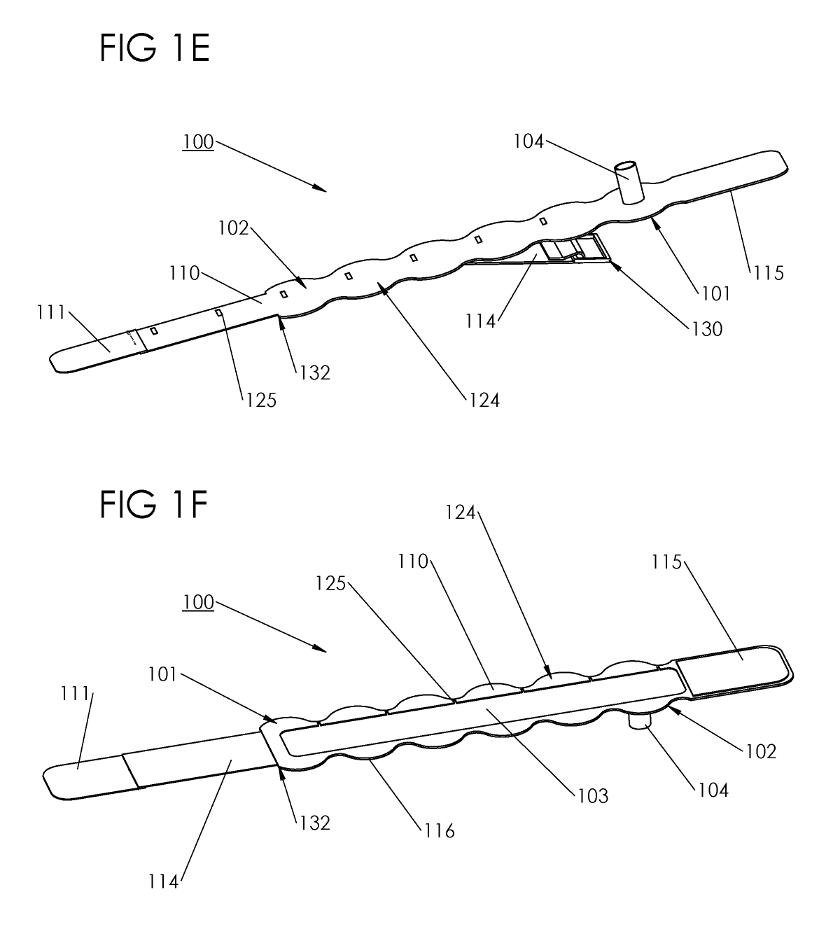

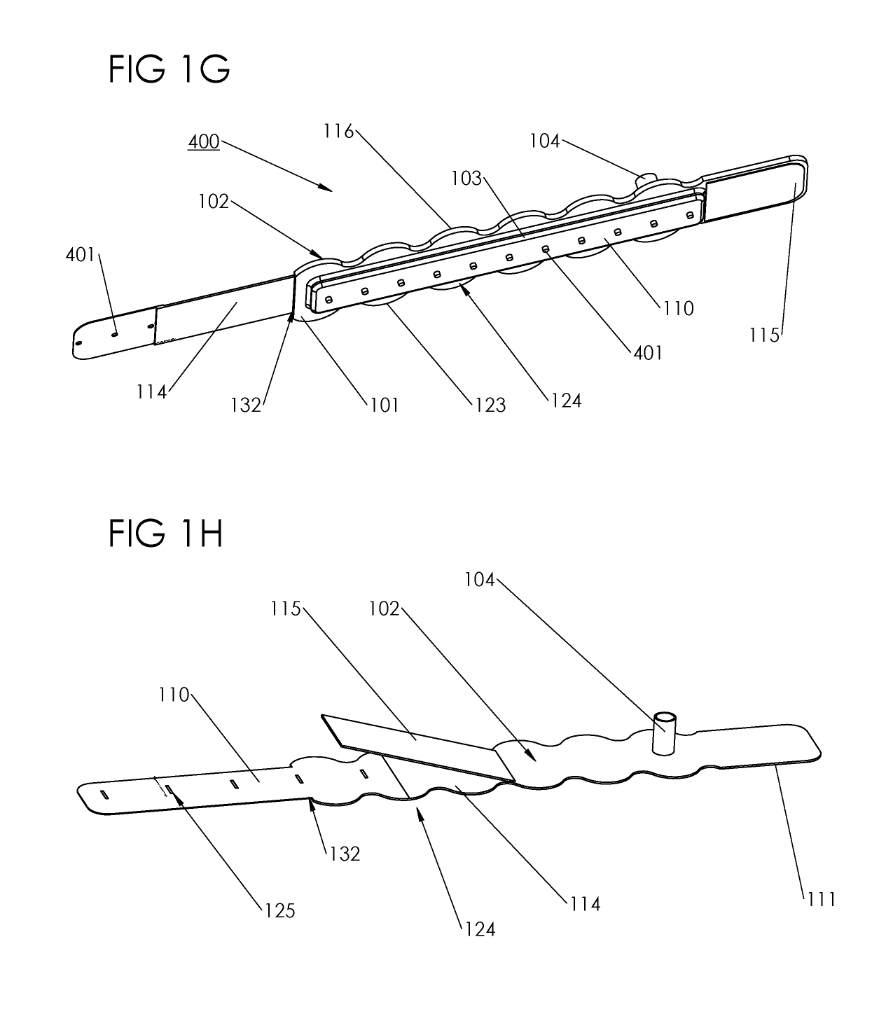

A preferred embodiment of a belt 100 for use in a BFR system is shown in FIG. 1A. The belt 100 is preferably inflatable and in the context of this preferred embodiment and alternate embodiments, the reader shall note the applicant is referring to inflatable belt constructions. However the reader shall not limit the scope of this invention to inflatable belt structures and shall understand that the inventive concepts and methods herein may be adapted for use with non-inflatable belts as well.

The belt 100 is comprised of an outer belt material 127 which is preferably substantially inelastic, or non-stretch, such as single or double side urethane coated ballistic nylon of 200 denier, or preferably a loop fastener laminated with a polyurethane coating, wherein the loop fastener and polyurethane coating are integrally formed. Such class of material is commonly referred to in the fabrics world as non-stretch, and where the term inelastic is used in this application, the reader shall understand the applicant's intent is to refer to this non-stretch class of material. The reader shall further understand that non-stretch fabric is understood in the industry to have certain characteristics regarding stretch, such as in the warp and fill directions, and other materials which may not necessarily be considered fabrics, but that have similar non-stretch properties shall also be considered “non-stretch” or “inelastic” within the context of this application. For the purposes of this application, non-stretch materials may be assumed to have a stretch factor of greater than 40 lb/in.

The reader shall understand that material properties of the belt may be changed to alter the compressive response to the limb as described in the operation section of the preferred embodiment. For example, a stiffer material may provide a harder, stronger response, and a more elastic material may provide a softer attenuated response. This may be advantageous in designing the belt 100 for different types of users. For example, those with big strong limbs may desire a stronger, harder response while the elderly or frail may desire a softer response from the belt 100. The strength/weight of the fabric may be lighter or heavier, such as 50 denier or 800 denier, and lighter fabric may provide additional advantages in terms of cost, weight, and compliance for conforming to the body. Preferred aspects of the outer belt material 127 are: it doesn't stretch or stretches to a very small degree, can hold a gas, or is substantially airtight, and can be connected in an airtight fashion to an inner belt material 126, forming at least one inflatable chamber 103, and optionally at least a second inflatable chamber 117. In the context of this application, the reader shall understand that an inflatable chamber 103 shall refer to a distinct zone or pocket on the belt 100 that is designed to deform into a specific shape under inflation and may be connected to other such inflation zones via airtight pathways. In the context of FIG. 1A for example many interconnected inflatable chambers 103 are illustrated, and each one of these sections shall be considered an inflatable chamber in and of itself. Additionally in the case of FIG. 1F, an inflatable chamber 103 is elongated and spans the length of the belt 100, and there are no additional chambers to interconnect with. This elongated inflation zone shall similarly be considered an inflatable chamber 103. The quantity and sizing of chambers may be changed depending on the desired length of the belt 100 and desired range of limb sizes the belt must cover, and whether the concept of targeted compression zones will be used as discussed later, but it is preferred there are at least two inflatable chambers 103 as shown in FIG. 1A, which shows six chambers. The reader shall further note that while a multiple chamber belt as described in pending application Ser. No. 15/428,141 to the applicant, is a preferred construction, all invented concepts and methods disclosed herein may be similarly adapted to a single elongated chamber belt as shown in FIG. 1F, or as disclosed in Sato and commonly seen in design of blood pressure cuffs.

The outer belt material 127 may be machine washable. A substantially inelastic outer belt material 127, and in particular as configured in the multi-chamber configuration of FIG. 1A, removes the need for complicated and expensive limiting plates and other such constructions as described by Sato. The reader shall note that the outer belt material 127 could also be elastic, it would just be less effective because inflation of the chambers would cause the overall size of the belt to expand outward away from the user. Additionally, limiter plates, or alternatively a strip on inelastic fabric, could be added around, or fixed to the elastic outer belt material 127 to limit expansion past a certain degree without departing from the spirit of this invention, but this is not necessary. The connection between the outer belt material 127 and the inner belt material 126 is preferably heat sealed, or RF welded, molded as in FIG. 1G, or as otherwise described in the prior art, however the reader may note that many means for attaching two fabric like materials in an airtight fashion, such as bonding, may be considered within the scope of this invention. The width of the outer belt material 127 may be approximately 1 in-3 in for belts 100 intended for the arms and approximately 2-4.5 in for belts intended for the legs. However, the reader may note that, as described in other embodiments in the referenced applications, the shape may also be non-rectangular and may span a wider or narrower region at different points around the circumference and these modifications previously described may be employed in conjunction with the pre-tensioning system disclosed herein. In general, for areas where freedom of movement is needed a narrower section may be beneficial, and for areas where a directed compressive load needs to be applied, a wider section may be beneficial.

The inner belt material 126 may be an elastic material or preferably an inelastic material, and may have a significantly higher degree of elasticity than, or identical elasticity to, the outer belt material 127. The inner belt material 126 may additionally comprise a loop fastener such that it integrally forms the first fastening means as well as the bladder wall. In the case of bigger more muscular limbs, the inner belt material 126 being elastic may allow the inflatable chambers 103 to bulge more into the limb and provide a better compressive action for the user. The reader shall understand the applicant contemplates mixing and matching materials and material properties to alter the behavior to best suit the user of the belt 100. The inner belt material 126 is preferably also washable, but not necessarily so. The inner belt material 126 similarly may be connectable to the outer belt material in a substantially airtight fashion, and may itself be made of a substantially airtight material. For example the inner belt material 126 may be polyurethane coated nylon stretch fabric, or may be exactly the same material as the outer belt material, providing fewer amount of different parts and therefore better economies of scale in fabrication. Together the inner belt material 126 and outer belt material 127 form a main portion of the belt 100 herein referred to as the belt body 124, and the reader shall note that the ends of the belt body may be extended to one or both ends and in a direction away from any inflatable chamber 103, but that these sections may still be considered part of the belt body. In the case of this preferred embodiment, the belt body is formed by two pieces of integrally formed loop fastener with polyurethane coating so that the bladder itself is integrally formed to comprise both the chamber to receive a gas as well as the fastening means as described later as necessary to apply proper pre-tension and a substantially non-stretch outer layer to allow for inflation and subsequent compression of the limb. The applicant is unaware of any prior art bladders that hold a gas airtight and are configured to be integrally formed.

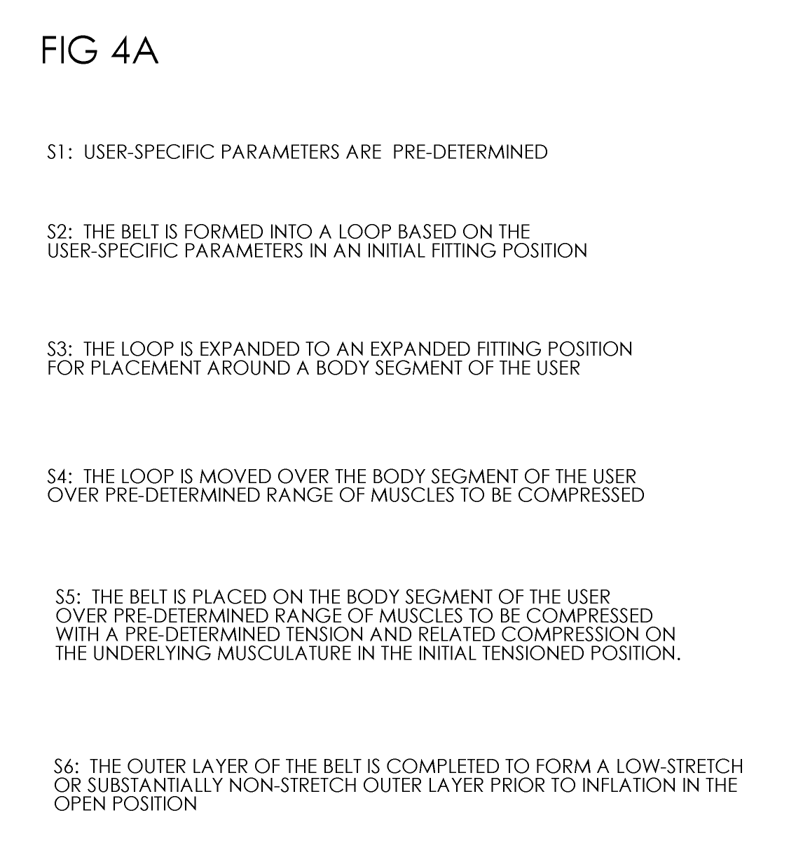

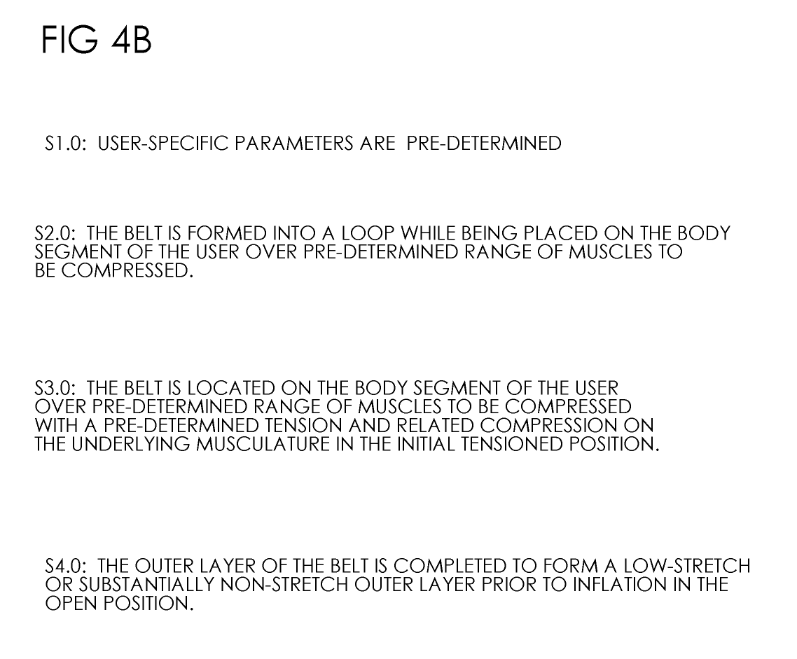

When the outer belt material 127 and inner belt material 126 are connected they form a series of interconnected inflatable chambers 103 as shown in FIG. 1A. The reader shall note that the lines shown to form the series of chambers are simply for illustration purposes so it is clear to the reader what the chamber pattern might look like. The connection profile forming the inflatable chambers 103, may be along the entire length of the inner belt material 126 or outer belt material 127 as shown in FIG. 1A, or may be along only a portion thereof, forming clusters of inflatable chambers as seen in Ser. No. 15/428,141, FIG. 1I. An inflatable chamber 103 that is formed along a portion of the length of the belt 100 may have the advantage that it only applies compression to a specific region on the limb and thus reduces the overall sense of compression to the user, improving a feeling of comfort. The interconnected inflatable chamber 103 also need not be of equal size. The inflatable section of the belt may have a non-rectangular profile comprised of cylinders of different sizes as shown further in Ser. No. 15/428,141 in the embodiment of FIG. 4. The inflatable chambers 103 may be located anywhere along the length of the belt 100 and are not restricted to starting near one end, for example as shown in FIG. 1B on the left end.

The diameter of a cylinder and the ratio of cylinder length (height 120 in FIG. 1D) to cylinder diameter—known as the cylinder aspect ratio—are two important shape factors that affect the performance of the belt system as described in this application in the case of a multi-chamber design. It shall be noted that for the purpose of description the applicant idealizes the design in terms of a right circular cylinders in the fully inflated unconstrained state, whereas in fact the sealed ends of each chamber constrain the ends and prevent an inflated cylindrical shape at each end, the chamber only approaching a right circular cylinder at the midpoint along the length. With this understanding in mind, the design concept of a ratio of chamber height to non-inflated width and inflated cylinder aspect ratio of height to diameter remains valid.

Through theoretical considerations, experimentation, and practical considerations described in greater detail below, the applicant has arrived at ratios that work very well. The inflatable chambers 103, shown in their non-inflated state, have a height 120 to width 121 ratio (height/width) of, but not limited to, approximately 3:2. Understanding that actual inflated shape differs from idealized shape, this corresponds ideally to a cylinder aspect ratio (length/diameter) on inflation of approximately 2.4, meaning the inflated chamber length (length being substantially equal to height 120) is about 2.4 times longer than the diameter when fully inflated. The fact that the idealized shape and actual inflated shape differ slightly in aspect ratio is of no material consequence. Therefore, as stated above, we will describe our invention in terms of idealized shapes.

The height 120 and width 121 may vary in size and ratio without departing from the spirit or scope of this invention. For example the height to width ratio may be 9:8 on the low side to 6:1 on the high side and still achieve some amount of shrinking capability. The reader shall note that the height 120 and width 121 referred to herein, and as marked by the reference numerals, are for un-inflated dimensions and not for inflated dimensions. As discussed elsewhere in this application, the reader shall note the effect of increasing this height-to-width ratio reduces the end or edge effects as described in Ser. No. 15/428,141 providing better shrinking and compression. The downside is for a given belt 100 width, a larger ratio means shrinking the width, and therefore adding more chambers, which increases the number of chamber connecting joints 129, which do not shrink. Chamber connecting joints 129 forming a greater percent of the overall belt length act counter to the goal of creating a maximally shrinking belt. Additionally, for a given length of belt and quantity of chambers, if the chamber connecting joints form a larger proportion of the belt, the diameter of each chamber, when inflated will be smaller and so the additional compression achieved from the pressure of the inflatable chamber 103 pushing on the adjacent portion of the limb 90 itself is reduced. Finally, when the radius of curvature of the chambers is small, it is less capable of producing significant belt tension which further reduces the elasticity component of the system.

A range of limb girths, girth being the circumference or perimeter of the limb at the point of application of the belt 100, may be accommodated with varying lengths of belts and chamber sizes. Limb girths ranging from 19 cm to 32.5 cm may correlate to chamber height 120 of approximately 3-4 cm and chamber width 121 of 2-2.7 cm; limbs ranging from 30 cm to 47.5 cm may correlate to chamber height of approximately 3 cm-5 cm and chamber width of 2-3.3 cm; limbs ranging from 42.5 cm to 60 cm may correlate to chamber height of approximately 4.5 cm to 6 cm and chamber widths from 3 cm-4 cm; limbs ranging from 55 cm to 77.5 cm may correlate to chamber height of approximately 4.5 cm to 7 cm and widths of 3 cm to 4.7 cm; and limbs from 72.5 cm and up may correlate to chamber height of 5 cm to 9 cm and chamber width of 3.3 cm to 6 cm. The reader shall note that these ranges are only some examples, but that dimensions stated herein may be increased or decreased without departing from the spirit of this invention. For example small arms of a child may use a chamber height of 2 cm whereas a body builder may use a chamber height of up to 10 cm for arms. For legs, a small child may use a chamber size of 3 cm whereas a body builder may use a size of up to 20 cm. These are but examples, and the reader shall understand that the applicant prefers a narrower belt for the benefits of avoiding complete stoppage of the blood flow, however such wider belt configurations may function with similar characteristics as narrower belts and as such, shall be considered within the scope of this invention.

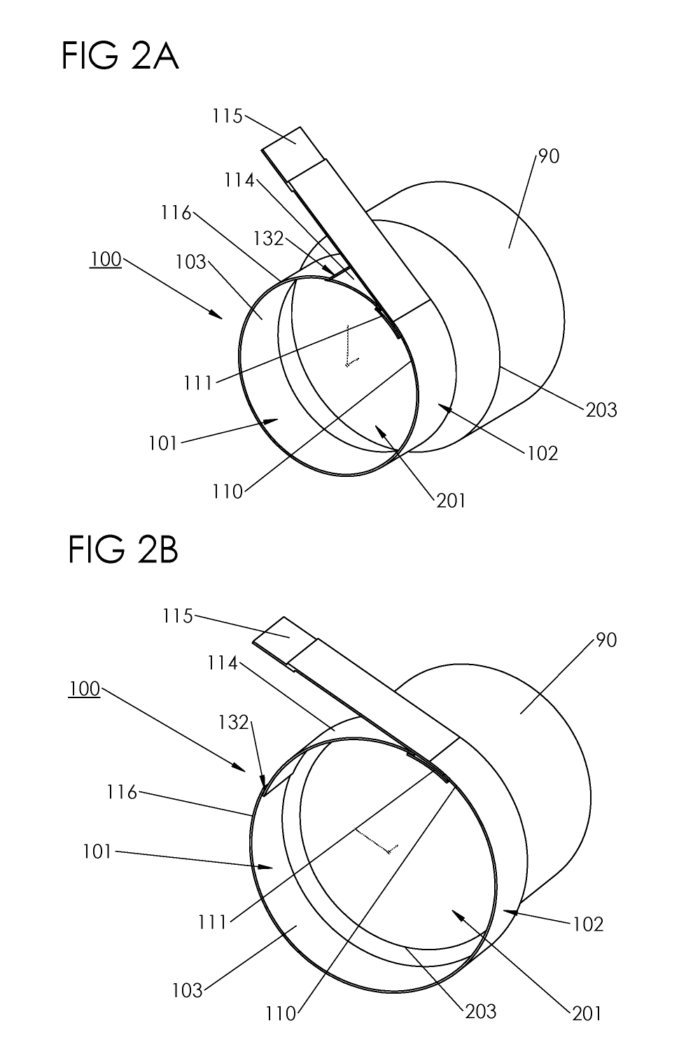

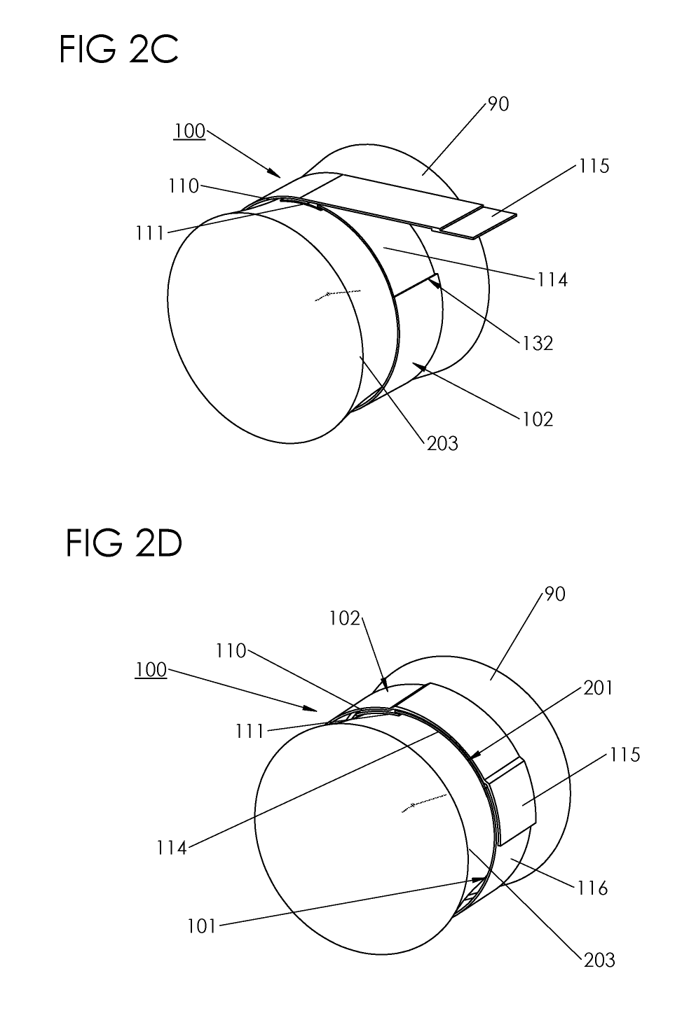

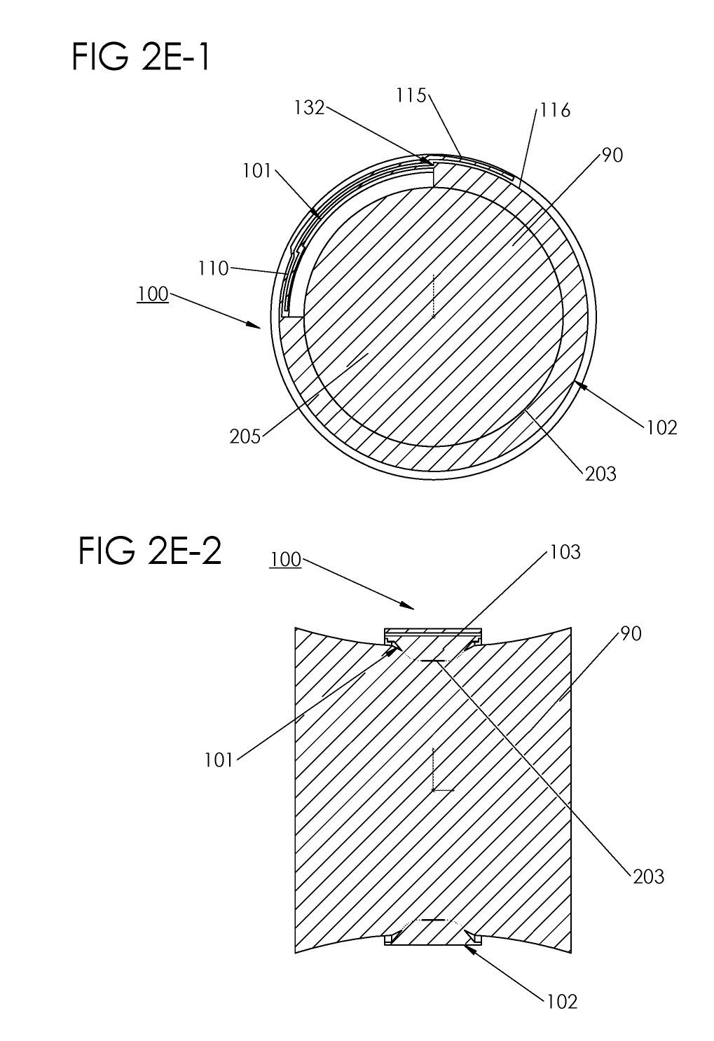

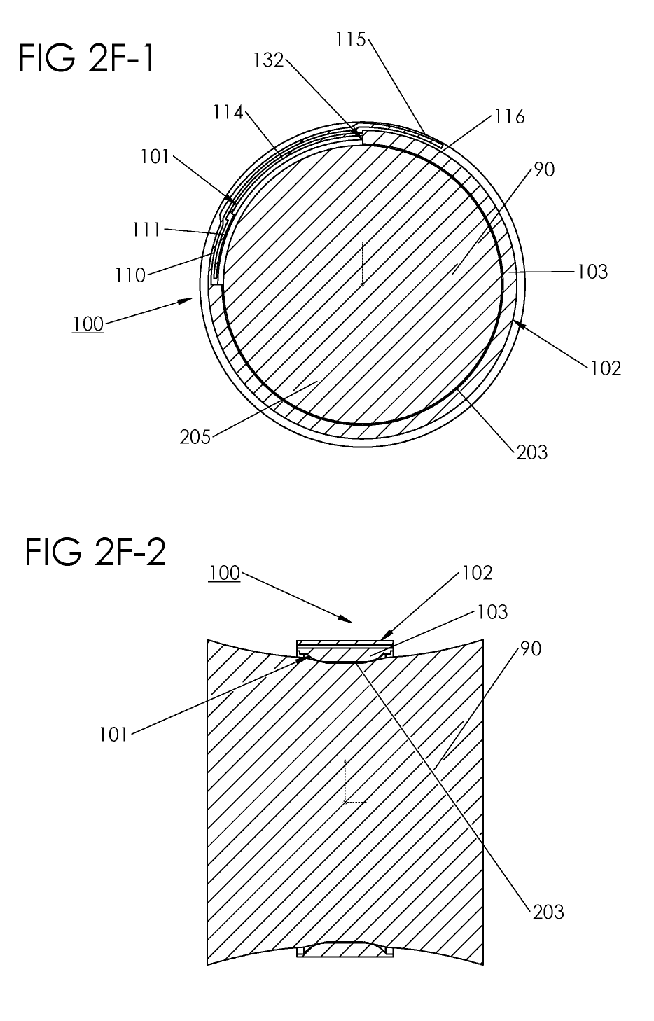

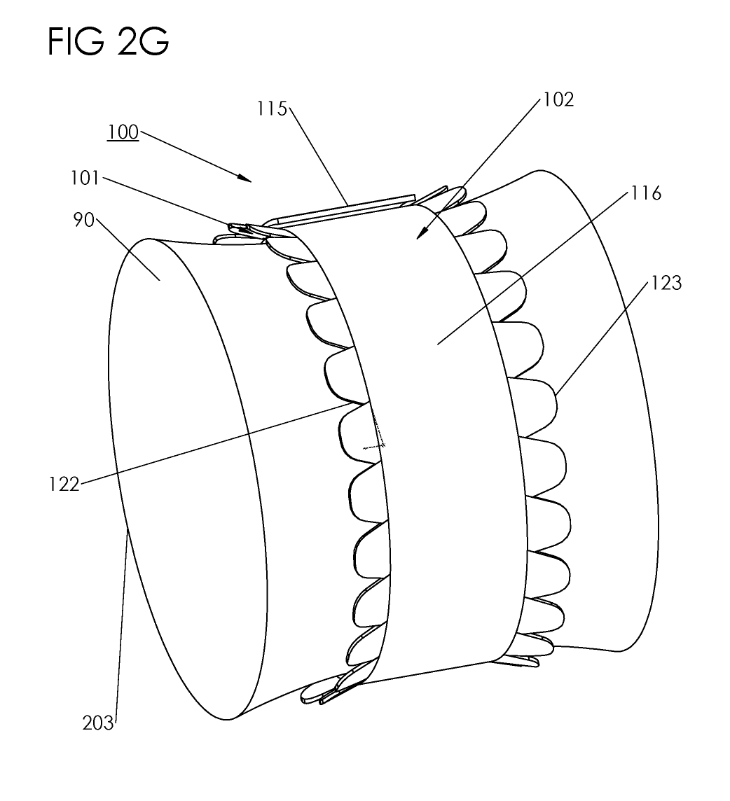

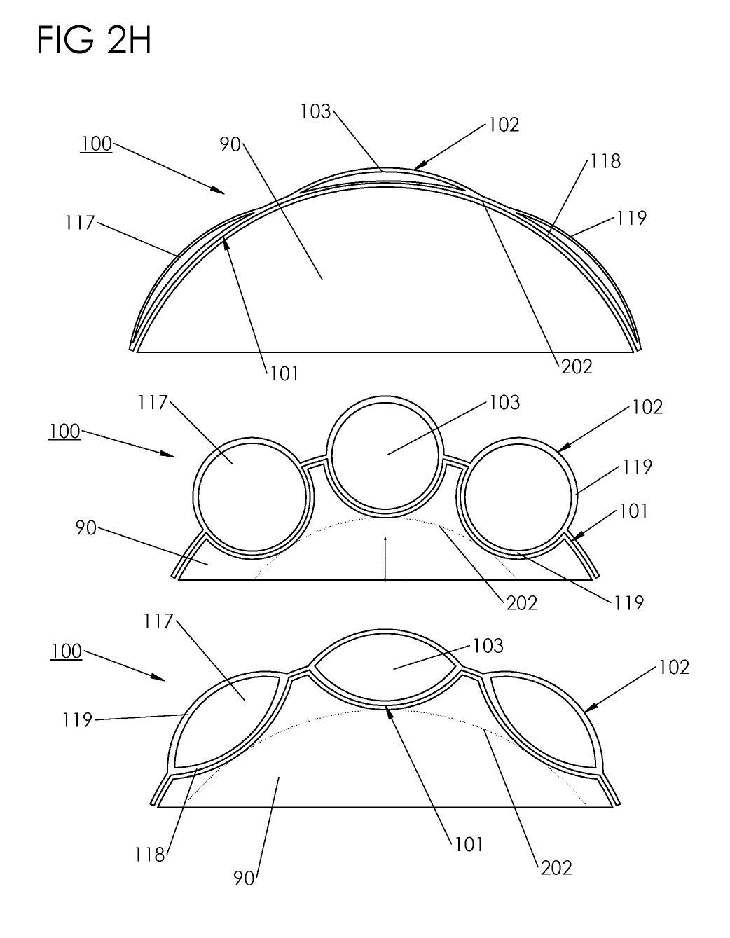

The inflatable chambers 103 may be interconnected as shown in FIG. 1A, or may not have a pneumatic pathway between them and instead have separate inflation sources. An advantage of the interconnection is a single inflation source is needed and air passes from one inflatable chamber 103 to the other. The chamber connecting means is smaller in width than the height 120 of the inflation chamber 103, and preferably connects the inflation chambers in the center of the belt 100, but may alternatively do so along any portion of the height of the inflatable chamber, including the ends of the inflatable chamber, or even on the width 121 of the inflation chamber. The connecting tube may be formed within the welding process, as in the preferred embodiment, or may be a separate component that connects each chamber. The connecting tube is preferably just small enough as to let sufficient airflow pass from one inflatable chamber 103 to the next without blocking airflow due do to inadvertent kinking of the belt 100 prior to or during inflation. There may be only one connecting tube or multiple connecting tubes to facilitate airflow. The corners of the connecting tube are preferably slightly rounded to reduce stress at this joint, and to facilitate opening and airflow during inflation and deflation. Experiments indicated that a connecting tube dimension of approximately 0.1875″-0.375″ wide and 0.1875″-0.375″ long works very well, although the reader shall note these dimensions are but examples and not meant to limit the scope of this invention. For example, a belt 100 that has targeted inflation as described in Ser. No. 15/428,141, may have a gap of 1 in or more between clusters of inflatable chambers 103 and therefore require a longer, and potentially wider connecting tube. The connecting tube is formed adjacent to at least one chamber connection joint 129. If the connecting tube is in the middle of the belt 100 as shown in FIG. 1A, then a chamber connection joint 129 is formed on either side of the chamber connecting tube. The chamber connection joint 129 (not shown in FIG. 2G) may have one or more optional cutouts 122 to allow the edge 123 of the belt 100 along the width 121 (not shown in FIG. 2G) of the inflatable chamber 103 to curl up and over a portion of the limb 90 as shown in FIG. 2G. The cutout 122 may be in the form of a semi-circle, or may be such that edge 123 is sinusoidal in shape as shown in FIG. 1A, or any other form that allows for additional compliance of the belt 100 for conforming to a limb 90. A curved, or hereinafter referred to as non-linear edge 123 was tested with users to evaluate comfort level as a sharp edge can scratch and irritate the skin if no clothing or other protective sleeve is worn underneath. McEwen also describes the importance of an edge protection mechanism, but does not contemplate a non-linear edge, and instead prefers to bind fabric edging around the perimeter. The purpose and aspects of the cutout 122 have been covered in Ser. No. 15/428,141 and shall not be repeated further here for the sake of brevity, except that the applicant's modification of the edge design improved comfort and flexibility as described below in the operation of the preferred embodiment, not only the ability of the belt to shrink.

However the reader shall note that while Ser. No. 15/428,141 describes edge effects and design in relation to a shrinking multi-chamber belt such as shown in FIG. 1G, the applicant's invention of a non-linear edge aids in the improved comfort of a single chamber belt, and in fact a non-inflatable belt as well as will be described in the operation section, and thus an entirely new benefit to the non-linear edge has been discovered. The reader shall note that the applicant means non-linear in that a curved belt, as mentioned in prior art conforms well to a conical limb, when curled around the limb forms a line around the limb and does not deform to accommodate edge effects. The applicant's definition of non-linear refers to the shaped of the edging when viewed wrapped around a limb. The reader may also observe the applicant's concept of non-linear includes an edging with one or more inflection points along the edge as can be seen in FIG. 1A.

FIG. 1A illustrates another example of how the inflatable chamber design 103 may be modified to improve the edge effects as described in Ser. No. 15/428,141, FIG. 1N which shows polygonal chambers. The reader shall note this is but one illustration and curved chambers, or other shapes may similarly be used without departing from the spirit of the applicant's invention. Similar to Ser. No. 15/428,141, FIG. 1N, the portion of edge 123 that is subjected to buckling is narrower and substantially shorter than inflatable chamber 103 width. Less material to compress and buckle means less of a counterforce and more effective for a given applied pressure. Edge effects have been extensively covered in Ser. No. 15/428,141 and shall not be repeated further here in the sake of brevity.

An input port 104 is in communication with the inflatable chamber 103 to allow a gas to flow into and out of the chamber. The input port 104 may be an RF weldable valve component, or simply a tube welded or heat sealed between the inner belt material 126 and outer belt material 127 as in an Intravenous (IV) bag. The input port 104 may protrude out one edge of the inflatable chamber 103 as shown in the previously filed patent 62/293,536, FIG. 1A-1, or may be connected perpendicular to the outer belt material 101 as shown in FIG. 1A. The input port 104 may be a straight, right angled, or slightly angled port. A port perpendicular to the belt 100 surface may have a benefit of being easy to connect an inflation means 106 (shown in FIG. 1H) versus a port that is parallel with the user's limb. The specific material and method of fastening is not critical as long as an inlet is created in an airtight fashion. The reader shall note that while not all embodiments show an input port, it shall be assumed that the belt 100 comprises a method of getting air into the belt for accurate control of the compression level on the underlying musculature. One or more valve configurations (not shown) may further be placed into the input port 104 as part of the belt 100, but this is not necessarily part of the assembly. Valve configurations have been extensively covered in the referenced utility applications to the applicant and be included fully here in reference as applicable to the applicant's invention disclosed herein. As discussed in other embodiments in the previously filed provisional referenced herein, there are many such valve configurations and combinations that produce beneficial results and the belt 100 of FIG. 1A may include any one of them, or none at all. The input port 104 may be placed anywhere along the length of the length 112 such that it is in communication with at least one inflatable chamber 103 to allow airflow into the belt 100. The input port 104 is preferably located, but not limited as such, on an inflatable chamber 103 on one end of belt 100 as shown in FIG. 1A, and preferably on the end that is wrapped around and overlaps the opposite end of the belt body 124.

The length 112 of the belt 100 may be comprised of an inflatable portion and a non-inflatable portion as described in Ser. No. 15/428,141. While Ser. No. 15/428,141 describes a fold-back style belt, the applicant herein is largely depicting and referencing a straight overlap style of belt 100 in this application. However the reader shall note that the concept of a portion of the belt 100 being non-inflatable, and this portion being placed over a target compression relief zone as described in Ser. No. 15/428,141, shall similarly be applicable to the applicant's invention disclosed herein. Where figures herein show lengths that of sections or components of the belt 100 that appear shorter relative to other lengths of sections of the belt 100, the reader shall understand that such lengths may be increased to make them comparatively the same or longer than other sections of the belt. In so doing, the reader shall understand that one skilled in the art may adapt the construction as depicted in the figures herein, to increase, decrease, widen, make less wide, or otherwise alter dimensions of the inflatable belt 100 herein disclosed without departing from the scope and spirit of the applicant's inventions.

The portion of the belt 100 that is non-inflatable is defined as any portion of the belt that does not comprise an inflatable chamber. The non-inflatable portion may be in multiple sections or one complete section. The reader shall refer to Ser. No. 15/428,141 in relation to the details of the non-inflatable portion and inflatable portion and their interaction in relation to a fold-back style belt as described in Ser. No. 15/428,141. Here, the applicant wishes to describe these relationships in relation to a straight overlap style belt 100 as depicted herein. The inflatable portion of the belt 100 may be determined relative to the range of the limb girths specified for each belt size. For each belt size, the minimum length of the inflatable portion may be sized such that for the largest intended limb, the inflatable portion covers the minimum percentage of the body segment circumference 203 required to provide adequate compression in a “target compression zone” as described in Ser. No. 15/428,141. A limb may range generally from 40 cm-100 cm for a leg and 15 cm to 50 cm for an arm. The body segment circumference 203 is the circumference at the location on the body segment 90 where centerline (or middle) of the belt 100 is to be placed and may change during operation due to contraction and relaxation of the underlying musculature. By example, if a range of limb sizes for a specific belt is 18 cm-35 cm, and the minimum desired percentage of coverage around the circumference is 50%, then the length of the inflatable portion would be 35 cm×50%=17.5 cm, corresponding to roughly full coverage of the smallest intended limb size for which the belt 100 is designed. The remainder of the belt length 112, the non-inflatable portion would thus be 35 cm minus 17.5 cm, or 17.5 cm plus some overlap length to secure the belt, and therefore a belt length 112 of slightly more than 35 cm. The applicant favors at least 40% limb coverage for reference.

Alternatively, limiting the maximum overlap of the inflatable chambers on the limb may be desired to limit the additional compression from multiple layers of inflatable chamber(s) and, in this case, the smallest intended limb girth for a particular belt size is used to determine the length of the inflated section. It has been shown that overlapping a belt on a limb adds more compression and at some point occlusion of blood flow is likely to occur. Therefore, a maximum allowable overlap of the inflatable portion of the belt may be desired. For example, if a 50% overlap of the inflatable portion is the requirement for maximal overlap, then, using the example above of a minimum limb size of 18 cm, the inflatable portion for the above belt size would be 18 cm×1.5, or 27 cm in length. By using the above minimum requirement of 50% limb coverage for targeted compression, this would correspond to a non-inflatable portion of the belt of 27 cm in length, and overall belt size of 54 cm in length 112. Possible limb sizes would then range from 18 cm-54 cm. The maximum allowed overlap favored by the applicant is 100%, twice around the limb.

As described above, the reader shall understand the method by which the belt components may be modified in length in order to design an appropriate belt 100 for a given range of muscles to be compressed and percentage of coverage over a target compression zone, and additionally when taking into account maximal overlap limitations for the smallest intended limb size. The reader shall note the guidelines for percentages of overlap and coverage are merely illustrative examples and that the applicant has previously described some of these aspects in detail in the referenced applications. Relating to maximum overlap coverage the application prefers to keep the overlap less than 100%, meaning that the belt would not wrap more than once around itself when placed on the limb while covering at least 40% of the largest limb.

A first fastening means 110 is disposed along a portion of the length 112 of the belt 100 as shown in FIG. 1A, and in this case, along an inner face 101 of the belt. Alternatively the first fastening means 110 may be disposed on an outer face 102 as described later in the alternate embodiments. The reader shall note that inner face 101 and outer face 102 refer to the general surface of the overall belt 100, or underlying components and not to a surface of any specific component. More generally, when the belt 100 is laid flat as in FIG. 1A, the inner face 101 refers to the side of the belt that will be laid on top of the limb and the outer face 102 refers to the side of the belt that will be exposed and away from the limb. The first fastening means 110 may be disposed along the entire length 112 of the belt 100 or only a portion thereof, and may be one continuous section or multiple sections. The first fastening means may further be integrally formed with the inner belt material 126 as is the case with a loop fastener laminated with a polyurethane coating. The preferred example for first fastening means 110 is a loop fastener, but the first fastening means may also be a hook fastener, adhesive glue, a button snap system, hooks like in a bra strap, or any other method of adjustably fastening to portions of the belt 100. FIG. 1A shows first fastening means 110 only on one section of the belt 100 but the reader shall note this is for illustration purposes only and in the case the first fastening means is integrally formed with the inner belt material 126, the first fastening means may be disposed along the entire length of the belt body 124.

A spring element 114 is optionally provided in FIG. 1A on one end of the belt 100 and may be connected via sewing, welding, gluing, or other suitable means known to those skilled in the art, otherwise integrally formed with either or both of the inner belt material 126 or outer belt material 127. The spring element 114 may be a stretch webbing or other spring like material. The spring rate of the spring element 114 may be, and generally is desired to be, substantially lower than the spring rate of other components in the belt 100, such that when a loop 201 is formed with the spring element as shown in FIG. 2A (as will be described in the operation), the loop has the ability to stretch substantially relative to when the belt is completed in the open position of FIG. 2D, forming a substantially non-stretch or high spring rate “outer layer” that covers the spring element (as will be described in the operation). For example the spring rate of the belt 100 in the open position may be at least 2×, but may also be 10×, 100×, or even higher the spring rate of the belt in the initial tensioned position.

The reader shall note that one difference with the applicant's invention and the prior art, including the applicant's own previously filed applications, is the optional introduction of a substantially elastic portion of the belt 100 for pre-tensioning purposes prior to inflation, or otherwise prior to final tensioning in the case of a non-inflatable belt. This low spring rate, elastic portion is more elastic as compared to the final belt form (the open position) which must form the belt 100 into an outer layer of much higher spring rate, or non-stretch entirely. Where the applicant discusses suggested spring rates of various components, the reader shall understand these are only suggested ranges and as long as the relative elastic difference between the initially tensioned state (prior to formation of the open position), and a ready-to-inflate state (the open position), or otherwise final tensioned state in the case of a non-inflatable belt, is preserved, such configurations and spring rate values shall fall within the scope of the applicant's invention for pretensioning.

Spring rates for the spring element 114 may be for example, but not limited to, between 0.5 and 20 lb/in for example, and spring elements may also be removable, adjustable, or interchangeable to modify the characteristics for different users. This lower spring rate is in contrast to the applicant's prior description of a belt spring in Ser. No. 15/430,404 and related provisional applications to the applicant where the belt spring would require a much higher stiffness in order to provide adequate compression on the body segment. The requirement of higher stiffness of the belt 100 when wrapped in place around the limb 90 is similar in the case of Sato's designs where he calls for the belt itself to be stretchable, but still requiring a high spring rate since a low spring rate would not adequately compress the underlying musculature. In the case where the spring element 114 is adjustable, the length of the spring may be shortened or increased to change the amount of tension in the loop 201 for a given circumference. This may be done for example where the spring element 114 is comprised itself of an elastic hook and loop fastener where the length of the loop may be altered by fastening the hook and loop in a different place. A strap adjuster (not shown) may similarly be used to adjust the length of a spring element 114 and the reader shall note there are many ways known to those skilled in the art to adjust dimensions of strapping. Additionally hooks like on a bra strap may be used to connect one end of the spring element to itself in different locations to change the effective length of the spring element 114. The reader shall understand that there is a limit as to the appropriate initial tension that should be applied to a user prior to inflation from a comfort standpoint and that this maximum may be, but is not limited to, between 10 and 20 lbs of tensile force. This maximum tensile force may be developed by the stretch in the spring element 114 or if no spring element is present, by the belt body 124 itself when formed into the loop. The reader shall note that many such methods of modifying a spring element are known in the art and shall be considered within the scope of this invention.

A second fastening means 111 is depicted in FIG. 1A at one end of the belt, and is preferably hook fastener in the configuration of FIG. 1A. The second fastening means 111 may be connected to one end of the spring element 114, if provided, as shown in FIG. 1A via suitable means such as sewing, welding, gluing, etc. The second fastening means 111 may alternatively be formed of such material that it also forms the spring element 114 thereby reducing one component in the system. The second fastening means 111 may be other than hook fastener and may be loop fastener, button snaps, etc. The only important aspect is that the second fastening means 111 mates with the first fastening element to form a loop 201 as shown in FIG. 2A, which dictates the pre-tension setting of the belt.

The reader shall herein note that the spring element could be omitted from the design at this point and the belt still allowed to form an intermediate “adjusting” state be attaching second fastening means 111 to first fastening means 110 and forming a loop around the body segment 90. There would not be a substantially elastic component in the system at this stage, but since the pretension levels are not required to be high, the belt may still be movable on the limb for proper positioning. This belt wouldn't have the benefits of being insensitive to limb girth, but it would provide a beneficial positioning state that does not exist with prior art and will be described later in the operation section.

The reader shall further note that the attachment of second fastening means 111 to first fastening means 110 in turn secures the position of the loose end 132 of the belt body from moving relative to the limb and resists lateral movement as well from slipping out from under the portion that will overlap.

Optional markings 125 may be disposed along the length 112 of the belt 100, which indicate a desired pre-tension setting for the belt. The markings 125 may be lines printed on the fabric, or notches, other similarly visible feature for the user to recognize. The markings 125 may be permanently attached to the belt 100, or may be temporarily attached, for example with hook and loop fastener. The markings 125 may be provided in a plurality of markings, or presented only as a single, movable marking in the case of markings not permanently attached to the belt 100. The markings 125 may be only on one component of the belt 100 or on multiple components of the belt 110 as shown in FIG. 1A, or alternatively on a separate strip of fabric for example, sewn, or otherwise attached, to the belt along the length 112 where it is critical to have markings.

Belt 100 preferably further comprises a third fastening means 115, and an optional fourth fastening means 116, which are used to lock an outer layer of the belt 100 in a substantially stiffer or non-stretch configuration than the loop 201 formed with the spring element 114. The tension in applying third fastening means 115 in the belt therefore is equal to or greater than the tension was when applying second fastening means 111 in the initial tensioned position of FIG. 2C. Depending on the configuration (as will be further illustrated in alternate embodiments), a fourth fastening means may or may not be necessary. The third fastening means is also not required, but would lead to a less effective system as stretch in the outer layer would be significant and not apply substantial muscle compression. Nonetheless omitting the third fastening means 115 could be done and therefore should be considered as one derivation off the preferred embodiment.

Third fastening means 115 is preferably a hook fastener, or equivalent fastening means as previously described, and is in communication with one end of the belt body 124. The third fastening means 115 may be attached via sewing, welding, or other method known to those skilled in the art and may protrude off the end of the belt body 124 or use the belt body, inner belt material 126 or outer belt material 127 as a backing. While the length of the third fastening means 115 is depicted in FIG. 1A as being relatively short compared with the belt body 124 the reader shall remember the applicant has already discussed how component lengths may be altered in order to increase the range of applicable limb sizes the belt 100 may be used for.

The optional fourth fastening means 116 may be disposed on the outer face 102 of the belt 100 as shown in FIG. 1A and may be along the entire length 112 of the belt 100 or only a portion thereof as shown in FIG. 1A. The fourth fastening means may be similar to the first or second fastening means in properties and fastening method, it must only mate with the third fastening means. In the configuration of FIG. 1A, the fourth fastening means 116 is necessary for the third fastening means 115 to have a place to attach, but the reader shall note that other configurations as shown in later embodiments do not require the fourth fastening means, and rather use the first fastening means 110 for the same intended purpose, thus saving a component.

The length of the spring element 114 may be such that when it is formed into a loop 201 as shown in FIG. 2A it can provide enough pre-tension to adequately restrict the muscles in the smallest position for which the particular belt 100 is designed. Therefore the length of the spring element 114 (and potentially in combination with the second fastening means 111 depending on the configuration and connection technique), denoted by L2 in FIG. 1A, hereinafter referred to as the spring length 113, are preferably smaller than the smallest circumference to be compressed. In FIG. 1A, in this situation, the spring element 114 and second fastening means 111 themselves wraps around the range of muscles to be compressed and provides a minimal amount of pre-tension in this scenario. By example, if the range of muscles to be compressed is 18 cm-35 cm in circumference, the spring length 113 would be less than 18 cm in length. The other factor in determining the length of the spring element 114, thereby the spring length 113, is the spring constant of the spring element 114, the amount of pre-tension that is required in the smallest case, and the length of the second fastening means 111. If, for an 18 cm limb, the desired pre-tension is 5 lb, the spring element 114 has a spring constant of 5 lb/1 cm, and the second fastening means 111 has a length of 2 cm, then the maximum length of the spring element is preferably 18 cm minus 1 cm (equating to 5 lb of force) minus 2 cm (the length of the second fastening means), or 15 cm. This is but one example and the reader shall note that many lengths and configurations are possible. The provided example is for illustration only of the applicant's preferred design guidelines when choosing lengths and configurations or components. If for example the spring element 114 is made of a material such that it may fasten to itself, such as elastic loop fastener, then the length of the spring element may be increased beyond what is described and still be able to form a loop 201 smaller than the smaller range of muscles to be compressed. All such variations of lengths and material properties the produce the applicant's intended outcome of a pre-determined pre-tension for a given limb size shall be considered within the scope of this invention.

Other modifications to construction, material size, etc. may be used to alter, improve the function of, or otherwise eliminate components from the belt 100. For example, if the inner belt material 126 is made wider than the outer belt material 127, the first fastening means 110 may only be attached to the inner belt material. The reader may note this is one example of the many combinations and possibilities of combining components, varying their sizes etc, and all such configurations may be considered within the scope and spirit of this invention. For example, the first fastening means 110 may overhang one end of the belt body 124 and if the first fasting means has an elastic property, it may also form the spring element 114, thereby eliminating a separate spring element component. The reader shall note that while various dimensions and ranges have been suggested herein, these by no mean shall limit the scope of this invention as additional sizes, or few sizes with less overlap may be perfectly suitable as described herein. Nor shall the inventions be limited to the overlap constructions depicted, but other straight-style belts, or other configurations may similarly be adapted to take advantage of the applicant's inventions.

An optional coloring element 128 may be coupled to the end of the third fastening means 115 or the belt body 124. The coloring element is preferably visible from either the inside, the outside, or both and corresponds to a specific size of belt. Reflective properties add the benefit of showing up at night for safety reasons if exercising outside, for example, on the street. The optional handle (not shown) as a loop may be any form that is easy to grab for tightening and loosening the belt 100, and may be in communication with one end of the belt where the third fastening means 115 is located.

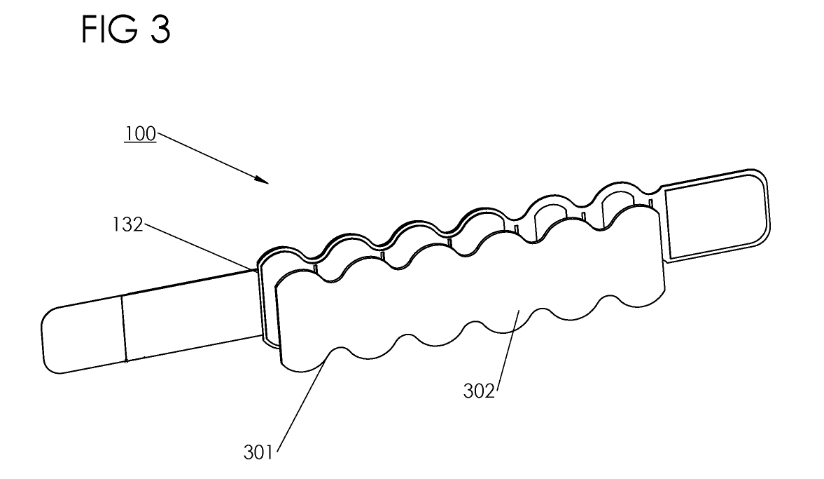

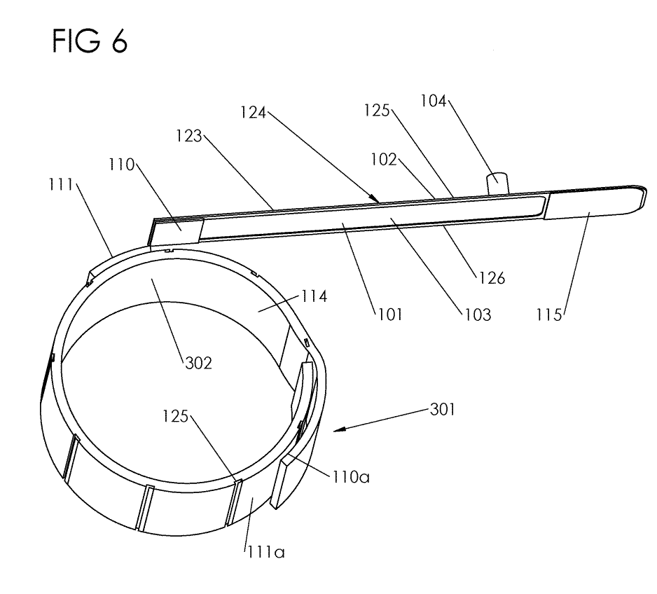

An optional body interfacing component 301 may be provided in communication with the inner face 101 as shown in FIG. 3, or as a separate member as in FIG. 6 that may be optionally connected to the belt 100 by suitable fastening means. The body interfacing component 301 is not required for sufficient functional operation of the belt of FIG. 1A, or other embodiments for that matter, but can offer some distinct advantages. As depicted in FIG. 3, the body interfacing component 301 may be a strip of neoprene closed-cell foam rubber similar to wetsuit material, approximately 2-3 mm thick, but may be as thin as 1.5 mm. Strips up to 5 mm thick were tested and all provide sufficient properties as herein discussed. The body interfacing component 301 preferably has a high friction surface 302, or in the case of the neoprene rubber, a “skin” side that is faced inward to contact the user. This high friction surface 302 may grab the user, or user's clothing such that rotation during initial tensioning, and slip along the limb during exercise, is not observed. Further, the body interfacing component 302 spaces the inflatable chambers 103 off of the limb, in the case of FIG. 3, by 2 mm. This gap between the inflatable chambers 103 and the user's limb (not shown) prescribes a certain additional volume that the inflatable chambers may inflate into as the inflatable chambers begin to compress the skin on the user's limb. In the case of FIG. 3, where the body interfacing component 301 is neoprene closed-cell neoprene foam rubber, the inflatable chambers 103 will compress and squish the foam material directly underneath, and while the full volume filled by the neoprene is not vacated, enough of it is deformed to allow for substantially more air to fill into the inflatable chambers than if the belt were placed directly on the user's limb without the body interfacing component 301, and direct skin compression is dissipated.

The body interfacing component 301 is further useful to facilitate donning of the belt 100 because, should the user want to employ this concept of spacing the inflatable chambers 103 off the surface, without the body interfacing component, there would be no friction or connection to the body (the gap would be filled with air) and this would cause the belt to slip down on the arm, and be very difficult put on. This phenomenon was demonstrated in experiments leading to the invention of the body interfacing component 301. The body interfacing component 301 may be fixed to the belt 100, and even double as the spring element 114, or may be separate as shown in FIG. 6. In the case the body interfacing component 301 is separate, the body interfacing component may comprise a body interfacing first fastening means 110a,b as shown in FIG. 6, and may be independently placed around the range of muscles to be compressed. The body interfacing component 301 dimensions may be adjustable as shown in FIG. 6, such that the body interfacing component itself provides the pretension on the limb and additionally serves as an anchor for placing the belt 100. The first fastening means 110 of the belt 100 may connect to a second fastening means 111, disposed on the surface of the body interfacing component, and the belt further applied around the limb as described later to form a substantially non-stretch outer layer. In this way, the pre-tensioning invention is preserved even though the pre-tension aspect is not integrally formed with the belt 100.

The body interfacing component 301 further helps to guard the user's skin against pinching. Pinching is a result of kinking in the case of a single inflatable chamber 103 that can be observed in the previously filed provisional 62/293,536, FIG. 1L, where there are kinks in a non-pre-stretched belt of that provisional filing. Sato's bladder designs have this kinking problem, and while the invention of a series of inflatable chambers ameliorates and even solves the kinking issues, the body interfacing component 301 further distributes the load and eliminates any chance of pinching the skin. This pinching phenomenon was another observed problem with Sato's equipment where Sato only contemplates a thin liner, and not something that will disperse and eliminate kinks. The body interfacing component 301 further serves to distribute the applied load from the gas bladder 103 across a certain width, and on to the user's limb. It is an important property of the body interfacing component that it be squishy to a sufficient degree so as to maintain a soft squishy interface to the user to provide maximum comfort, but be able to move and allow room for the inflatable bladder 103 to expand prior to compressing the user's limb.

The body interfacing component 301 may be permanently connected to the belt 100, or may be removable as shown in FIG. 6, or may be applied prior to application of the inflatable band, and not attached to the inflatable band at all. In the case the body interfacing component 301 is attached to the belt 100, such attachment may be done with sewing, bonding or similar means. If provided, the attachment means for connecting the body interfacing component 301 to the inflatable band 100 may be two strips along each side or may be a single flat strip across the entire width. In the case of a single strip however, since the attachment means is blocking the gas bladder 103, the attachment means should itself have similar squishy properties as the body interfacing component, or else it will block the air bladder from expanding sufficiently and compressing enough of the limb. Of course the use of hook and loop fastener is mainly discussed here because of its simplicity, but adhesive backed tape, magnets, or other non-permanent fastening means may also be used.

Further utility, features, connection means and configurations, etc of the body interfacing component 301 have been described in the previously filed provisional 62/293,536 referenced herein, and shall not be repeated for the sake of brevity. The reader shall recognize that all such features of the body interfacing component 301 benefit the inventions described in this invention, and may be added to the inventions herein to achieve similar benefits as to the previously filed provisional patent's inventions.

Thus it can be seen that, while optional to the function of the belt in providing BFR, the body interfacing component 301 can serve a variety of important roles to improve both the comfort, ease of use, and safety of the belt 100. The body interfacing component 301 may be of sufficient width, length and quantity to serve one or all of the above described functions, and does not need to fulfill all the requirements above to be considered valuable. The body interfacing component 301 may also aid in improving hygiene and cleaning, and if removable can be washed and replaced. Body interfacing component 301 is shown in FIG. 3 to conform to the shape of the belt 100, however, it may be of any shape such as a rectangular shape that covers the belt, and specifically the curved edge 123.

While the non-linear edge 123 previously described can solve the chaffing or scratching issue against the skin, optional edging (not shown) may also be placed around the perimeter of the belt 100 to finish the edges and provide a smooth, soft, interface to the body. Optional edging may be soft material such as felt, compliant, and may further be elastic. Elastic edging adds the benefit that it will contour better to the body and should the belt 100 incorporate cutouts 122, this elasticity may help such that the length of the edging may increase or decrease slightly as the edge diameter needs to stretch, for example over an expanding bicep. Should the optional body interfacing component 301, be used, the edging may capture the body interfacing component, the outer belt material 127 and the inner belt fabric 126, the first fastening means 110 and the further fastening means 111 all in a single stitch for example so that there is only one stitch used in assembling the construction. As described in prior art, the components, if properly coated may also be RF welded, or bonded together in a single operation. This improves cost savings and speed of manufacturing as sewing is the most labor intensive process. The potential cost savings are significant compared to Sato's construction which involves multiple connection steps, and more components. Should the body interfacing component 301 be used, the edging may leave one end open. The open end may be used to air out the inside of the pocket after washing in a washing machine or after swimming with the belts, thus avoiding bacterial growth. As the belt 100 may be used in a hospital or medical setting, sanitation and washing is a key attribute not contemplated or mentioned in any of the prior patents.

The applicant's preferred method of connecting the inner belt material 126 and the outer belt material 127 to form the belt body 124 is RF welding, and a die is created such that it welds the inner belt material 126 to the outer belt material 127 to create the seam profile as shown in the internal pattern depicting the inflatable chambers 103 with a single welding operation. Optional cutouts 122 are further depicted, and while belts 100 were built without such features and demonstrated to function fine, particularly around the legs, feedback showed that such features may be useful in allowing better contouring and more efficient radial compression, particularly around the arm. Such cutouts 122 may be on only one, or both fabric materials, and if the inner belt material 126 is elastic fabric for example, use of cutouts may not be necessary as the fabric is itself conforming and stretchy. The input port 104 is depicted as a straight angle connection, but may be right angle as well for insertion of a belt valve (not shown). The input port is preferably placed close to the third fastening means 115, but not limited to such location. There is a hole in the outer belt material 127 to allow a gas to flow through the input port 104 and into the inflatable chambers 103. Only one input port 104 is shown, however the reader shall recognize that in lieu of connecting tubes, multiple input ports may be used to inflate various chambers.

To fabricate the belt 100 of FIG. 1A, first the input port 104, the inner belt material 126 and outer belt material 127 may be RF welded together. In the preferred embodiment the inner belt material 126 and outer belt material 127 may be integrally formed loop fastener with polyurethane coating and thus this welding operation simultaneously forms the belt body 124, inflatable chambers 102 and disposes the first fastening means 110 and fourth fastening means 116 along the entire length of the belt. A portion of the belt body 124 may be cut to overhang one end, and on this overhanging portion, the third fastening means 115 may be RF welded to the belt body. On the opposite end of the belt body 124, the spring element 114 may be RF welded (or stitched), and to the end of the spring element, the second fastening means 111 may be RF welded (or stitched). This completes the formation of the most basic and elemental form of the belt 100. While, as previously mentioned, various elements could be combined, the reader shall note that these combinations shall all be considered within the scope of this invention and naming every possible combination or alteration in material property is not practical in the sake of brevity. Nonetheless the applicant considers all such modifications as contemplated by the applicant and therefore considers them within the scope of this invention.

One aspect of any inflatable belt 100 that has at least one portion which overlaps, such as prior art to Sato, is that the overlapped section wants to slip out from underneath the overlapping section when the sections of the chamber(s) are inflated on top of one another. This slippage is more prominent the narrower the belt 100 because the inflated cross sectional profile becomes more and more like a circle the narrower the belt. Thus the bottom bladder “circle” wants to slip out from underneath the overlapping bladder “circle”. This phenomenon occurred to the applicant during testing, and when the overlapped section slips out the side, the compression on the limb is lost and the training is ruined. This slippage problem is accentuated by dynamic movement of the limb and muscles expanding and contracting, which is the intended usage as a training device for the applicant's invention. As the overlapping sections are moving relative to one another the bottom starts to slip out, there is nothing holding it in place. Therefore, by securing the loose end 132 from moving laterally with the usage of the pre-tensioning and positioning system, and further securing the other end to the belt body 124, the applicant has not only accomplished the pre-tension and positioning benefits but simultaneously solved this fundamental problem with overlap style belts slipping out to the side, and becoming unusable. The reader shall note that the body interfacing component 301, if a fastening means is provided as shown in FIG. 6, can also accomplish this same goal of keeping the overlapped end of the belt 100 from moving lateral and losing compression during exercise. In this case the body interfacing fastening means 110a,111a on the body interfacing component 301 anchors the overlapped end of the belt to the body segment 90 and the lateral resistance of the body interfacing component itself ensures the overlapped end of the belt doesn't move.

The applicant wishes to point out that while this invention relates to the pre-tensioning aspect of a belt 100 for blood flow restriction training, the reader may refer to the applicant's referenced patents for additional information about the overall operation of the belt design for function in the field of BFR training. Whereas the applicant's previous inventions aimed to solve the usability deficiencies of the prior art for BFR training specifically during the exercise phase of process, the applicant's current invention aims to solve deficiencies in the prior art in relation to consistent, measurably, and prescriptive setup in position and tension of the belt prior to operation. The applicant's inventions as herein described have been depicted as related to a straight overlap style for a belt consisting of multiple chambers, but all these concepts can be adapted to either a fold-back style belt or a single chamber belt (as will be described later) without departing from the scope and spirit of this invention.

### Preferred Embodiment—Operation

The following description will discuss two methods of operation that both relate to the preferred embodiment and each of the alternate embodiments. While the applicant prefers the method of operation depicted in FIG. 4A, the reader shall note the most basic state to create is the initial tensioned position as shown in step 2.0 in FIG. 4B, which is created while putting the belt 100 directly onto a body segment. In FIG. 4B this is done by a user applying the belt 100 directly to the body segment 90 without forming a loop via attaching fastening means and stretching over the body segment. Therefore such a method of operation shall be briefly described and likewise considered within the scope of this invention. The reader shall also understand that when the initial tensioned position is created (FIG. 2C), the intermediate adjustable state the applicant has invented is created and the belt may be rotated or moved longitudinally on a limb easily. If a spring element 114 is provided in the construction, then a tension may also be more easily determined, but the applicant may recognize that, especially on the legs where two hands are free, it may be possible for a user to also determine a reasonable pre-tension without this elastic component, and then optionally apply additional tension needed and lock the belt down in creating the open position of FIG. 2D.

The belt 100 of FIG. 1A may be applied as follows in conjunction with a BFR system. Regarding the flow chart of FIG. 4A, first in S1, a user (not shown) selects an appropriate user parameter. A user parameter may be the body segment circumference 203 as shown in FIG. 2E-1 and 2E-2 intended for the belt, it may be an intensity level they desire, the strength of the spring element 114 they are using, or anything else that may be specifically related to the user or their belt 100 or related to the user in combination with another user parameter. For illustrative purposes assume the user parameter is the user's limb circumference when the underlying musculature is relaxed, body segment circumference 203, where the belt 100 will be placed. The user then takes this user parameter, relaxed body segment circumference 203, and may correlate it to a specific marking 125 other otherwise determined position on the belt 100 per the manufacturer's guidelines. The guidelines may be provided on an instruction sheet in table form, on an app, or otherwise made readily available to the user on a mobile device for example. In the case that multiple parameters are to be combined, for example an intensity level in conjunction with a limb size, the two parameters may be jointly used in determining which marking 125 is appropriate per the manufacturer's instructions. Such combination may be via an algorithm, table, chart, or other form known to those skilled in the art. The reader shall note that a marking may not be necessary, a simple measurement for example from one end of the belt 100 may be made.

Next in S2, the second fastening means 111 may be fastened to the first fastening means 110 according to the guidelines from S1. This forms the belt 100 into a loop 201 as shown in FIG. 2A. The applicant shall consider this position the initial fitting position. As previously mentioned the spring length 113 formed by the spring element 114 and the second fastening means 111 is less than the circumference of the body segment 90 where the belt 100 is to be placed. Because the spring element 114 is now formed into a loop 201, the circumference of this loop is also less than the circumference of the limb where the loop will be placed. Accordingly, the enclosed area 205 of the loop 201 is less than the cross sectional area of the body segment 90 where the loop will go. This dimensional relationship is critical as this dictates the amount of compression the loop 201 will place on the body segment 90 when the loop is placed over the body segment as well as anchors the loose end 132 to the body segment. In all prior art, the equivalent of the loop 201 is always LARGER than the body segment, not smaller when formed prior to placement on the body segment 90. Other than the case of the smallest limb, a portion of the belt body 124 may also make up the loop and as the intended body segment 90 circumference increases the belt body may form a larger percentage of the circumference of the loop. The maximum circumference of the loop therefore is the length of the belt body plus any additional length of the third fastening means as described in the preferred embodiment. The reader shall note that S1 is not a mandatory step to reach this point. A user may create the initial fitting position without markings or guidelines and just based on feel. While this may not be as accurate or prescribed, it is a valid form of the pre-tensioning method invention of the application. The user shall also remember that S2 itself is optional, but may be advantageous, particularly in the case of the arms where it is very difficult to wrap a belt on oneself. Creating the lop 201 prior to placement on the arm makes it possible to stretch, slide, rotate the belt 100 into the correct location and orientation at the right tension as will be described next.

Next in S3, the loop 201 may be stretched and expanded to what the applicant will refer to as the expanded fitting position as shown in FIG. 2B. The elasticity of the spring element 114 allows for this expansion. In the case of Sato's design where it is the belt itself that must be expanded, the belt is far too stiff (because it must be to apply enough compression when inflated) to perform this expansion step with any sort of ease. As shown in FIG. 2B, the expanded fitting position has the loop 201 with a stretch circumference that is now larger than the body segment 90 and may easily be slipped over the body segment. The nature of the spring element 114, and its spring constant determine how much force must be applied and as previously mentioned the spring constant may be removable, replaceable and/or adjustable depending on the user. Such adjustments or replacements may be factored into the manufacturer's instructions as to how to account for the provided user parameters. The reader shall note that S3 is also optional if the user will be forming the loop 201 directly on the body segment.

Next in S4, the loop 201, is placed over the body segment 90 and released as shown in FIG. 2C. The applicant shall consider this the initial tensioned position, and this position is the first mandatory configuration to create. The reader shall also note as discussed relating to FIG. 4B, that the loop 201 may be created while simultaneously putting the belt onto the limb to create the initial tensioned position. The initial tensioned position now has the belt 100 securely fastened to the user with a proper, prescribed amount of pre-tension on the skin. The pre-tension may not be enough as to sufficiently restrict blood in and of itself, but it has placed the loose end 132 of the belt body 124 where the spring element is connected to a pre-determined distance along the belt body that has been dictated by the elastic properties of the spring element 114, the marking if used on the belt where the spring element was fastened with the second fastening means 111, and the circumference of the body segment 90. The tension created in the belt 100 at this stage by attachment of the fastening means keeps the belt positioned on the limb from rotating and sliding as well as applying the designed initial compression. This is the intermediate adjustable state that is lacking from prior art where the final tension, position, and orientation must be created simultaneously with locking in the open position (discussed next). At this point, because the compression on the limb is low, the belt may be rotated or moved longitudinally to finalize the correction positioning and even if the length of the belt is very long, the user doesn't need to maintain tension in the belt because the belt is held in place on the limb.

The reader shall note that if the user is wearing a slightly thicker shirt one day, compared to no shirt at all, the benefit of using a spring element 114 will be that the spring element will stretch a little bit more and apply a slightly higher degree of initial tension on the body segment 90, and that the connection point of the second fastening means 115 and the loose end 132 of the belt body will be slightly farther apart. This will in turn provide slightly more initial restriction and make the training slightly harder. However, if the spring element 114 is sufficiently elastic and has a sufficiently low spring rate as the applicant describes, the amount of additional restriction is not substantial, especially when compared to a stiff belt like Sato's or the applicant's referenced applications. In those scenarios, even a 5 mm-1 cm increase in internal circumference would cause the training to become unbearable because of extreme sensitivity to initial tensioning methods.

Next in S5, the loop 201 may be adjusted or rotated as necessary to properly position the input port 104 and ensure that no portion of the belt that may chafe the body or interfere with the body is facing the body. The belt, being secured to the body segment 90 at this stage may be manipulated easily with a single hand and will hold position, making it substantially easier to adjust and get to the proper location vs. the prior art, especially on the upper body. This is true because the initial tensioned position is not a significantly high tension state, and if a spring element 114 is provided it may be stretched in order to provide additional slack to add in positioning. In addition to the rotational orientation, the belt 100 may be moved longitudinally along the limb to avoid coving muscles or nerves. As the belt 100 has some tension on the body segment 90, it will stay in place and constantly hold the desired tension on the limb and thus, even if the belt is quite long, a proper final tension may be achieved as both hands are free to finish the wrapping. The readers shall also note that in the initial tensioned state, the loose end 132 of the belt 100, the end being overlapped in S6, is secured to the belt body and resists from slipping laterally out to one side as well as resists shrinking in length when inflated. As the belt 100 attempts to shrink during inflation, it pulls the spring element more and the overall tension of the system increases on the body segment 90. By securing the loose end 132 with the second fastening means (optionally via the spring element 114) the loose end is kept from displacing circumferentially and reducing the amount of applied compression on the limb. It is preferable that the displacement allowed be at most between 5% and 15% of the starting circumference of the loop 201 in the initial tensioned position.

Finally in S6, the outer layer of the belt 100 is completed by laying down the remaining loose portion of the belt with the input port 104, covering the spring element 114 if provided, and completing an outer layer that is substantially inelastic in what the applicant refers to as the open position. It is important to note that once the spring element 114 is covered up, it contributes only nominally to the stretching of the system because the spring rate of the spring element is far lower than that of the outer layer of the belt formed during the action of fastening third fastening means 115 down. Therefore under inflation or muscle contraction the spring element 114 does not detract from the belt's 100 ability to apply compression to the body segment 90. Further, the reader shall note that because the pre-tension and geometry of the belt around the body segment 90 was already determined by the loop 201 size (which was prescribed and pre-determined based on the user parameters and properties of the spring element 114), there is no real need to apply any significant additional tension when laying the third fastening means 115 down against the fourth fastening means 116 to complete the outer layer of the belt 110 around the body segment. This means that that user does not have guess or subjectively “gauge” an applied tension force, they can simply lay the belt down easily, which is an action that is simple for any novice to do consistently.

Of course the user may apply additional tension at this stage if they want, and the pre-tension applied resists rotation allowing them to do this, but it is not a requirement of the applicant's invention, and in fact the applicant has endeavored to make this aspect of the method of applying a belt for BFR obsolete as it introduces subjectivity and error in consistency into the system. The reader shall further note that even if a marking system were to be used with prior art systems, those belt designs and elasticity are so strong that any small change in limb diameter correlates to a big effect on the level of restriction. As stated in S4, the geometry and properties of the applicant's invention may be configured to provide a situation that is insensitive to changes in the underlying circumference of the body segment because of the low spring rate of the spring element 114. The effect of this is manifested in S6. For example, if a slightly thicker shirt is worn, slightly more additional pre-tension would be applied and the ends of the belt body would be slightly farther apart, maybe 5 mm or 1 cm. As described in S4, this applies a little more compression to the body segment 90, but since no real additional tension is required to lay down the third fastening means 115, and since the markings 125 are not relative to the third fastening means, the third fastening means can simply be laid down and no appreciably different in initial tension, and consequently active compression under inflation, would be experienced by the user. Conversely, if the marking 125 were related to the third fastener, and the user had to struggle and pull a relatively non-stretch belt tighter by 5 mm or 1 cm, a significant additional compression would be achieved and has been observed in practice. Thus, markings alone may not be sufficient for providing a robust system for consistently applying pre-tension to a body segment 90. The reader shall further note that by securing the loose end 132 belt body 124 to the belt body itself, the user is able to prevent that end from slipping out laterally from underneath the belt body. The spring element 114, or alternatively second fastening means 111, provides lateral support to resist the tendency or ability of the loose end of the belt body to slip out because it is physically restraining that end via the second fastening means. Thus, even during dynamic movement, the belt body 124 will remain fully overlapping and compression won't be lost.

In relation to getting to the stage of S6, the applicant has shown an alternative approach in FIG. 4B that a user could follow to create the basic state, the initial tensioned position. S1.0 is substantially the same as S1 as previously described. S2.0 is a combination of S2-4 as previously described. S3.0 is substantially similar to S5 as previously described and S4.0 is substantially similar to S6.0 as previously described. As the reader can see, the main difference is that the user may forgo forming the loop 201 prior to application onto the body segment 90 and instead opt to form the loop directly onto the body segment, using the markings as previously mentioned to fix the position of the loop 201. Such a method may be easier on the legs for example where two hands are available and it's easier to apply directly vs, expanding a loop and stretch over clothes. The applicant's invention of a pre-tensioning system and method therefore is meant to encompass all such modifications or changes in order of the steps as included in the scope and spirit of this invention. The reader shall note therefor the advantages to creating the initial tensioned position in S4 in that the use is afforded adjustability and can position, rotate, and tension the belt 100 on the appropriate body segment 90, in a pre-tensioned state that is snug, but has some flexibility, and then when everything is just right, can close the loop and tighten down to prepare for inflation. If the belt is long, which is beneficial in reducing the number of sizes needed to cover all limbs, the belt is easily wrapped around the limb (arm or leg) and the correct and desired tension is easily maintained. The benefits of elasticity in this state not only allow adjustments, but also allow for a predetermined setting to be used without significant regard to slight changes in the underlying circumference to be enclosed due to clothing, hydration, or limb size differences at the time of application.

This concludes the main operation portion of the applications invention herein which is meant to improve upon the setup of the belt 100 for BFR training. The applicant will briefly describe the remaining operation of the system for the reader's benefit but the reader should note that they may consult the applicant's referenced applications or other prior art for further details.

In the S6 the belt 100 was in the open position, which is a position in which a substantially non-stretch, or if elastic with a high spring rate, perimeter has been created around the body segment 90 and underlying musculature. The applicant prefers a non-stretch belt and therefore the remaining discussion shall relate to such a construction but the reader shall note that the description herein may be sufficiently modified by those skilled in the art to relate to a belt with a high spring rate as described in Sato's patents.

Air, or other gas, is introduced into the inflatable chambers 103 through the input port 104. Mechanisms for belt valves, and shutoff valves and other such mechanisms for inputting and maintaining air pressure have been substantially covered in previous applications and will not be repeated here for brevity. As air in enters the inflatable chamber 103, the belt 100 will expand inward due to do the inelastic nature of the outer layer and inability of the fabric to stretch any larger. In the applicant's preferred design of a series of inflatable chambers 103, the belt 100 itself actually shrinks in circumference. FIG. 2H shows how the belt inner circumference 202 changes during the operation. In the open position, which was formed by step S6 in the pre-tensioning workflow of FIG. 4B, the belt 100 is substantially flat against the body segment 90 as shown at the top of FIG. 2H. As air is introduced, each inflatable chamber 103 starts to form a cylinder and is shown fully inflated in an exaggerated position as a full circle in the middle image. At this point the belt inner circumference 202 has shrunk significantly inward on the limb and thus has caused the desired amount of blood flow restriction. The reader shall note that this overall level of restriction is clearly a combination of the pre-tension settings and the inflated pressure. If the pre-tension settings were loose, such that a significant gap existed between the belt 100 and the body segment 90, then it is conceivable the inflatable chambers 103 would not even contact the body segment 90 even when fully inflated. The fully inflated position when the belt has reached the target pressure referred to as the closed position.

FIGS. 2F-1,2 show what happens during a muscle contraction. The underlying musculature contracts and the body segment circumference 203 increases and pushes the inflatable chambers 103 back toward the state of the open position, thereby increasing the pressure in the chambers and likewise increasing the belt inner circumference 202. When the muscle relaxes the belt shrinks back down to the closed position. The expanded position due to muscle contraction is referred to as the working position.

The belt continues to transform, expand/contract, between the closed position where the body segment circumference 203 is the smallest, and the working position where the body segment circumference 203 is slightly larger, throughout the BFR training until eventually air is released and the belt is pulled off the body segment 90. To summarize, over the lifecycle of an operation, the belt 100:
1) starts in a flat position,
2) is optionally transformed into a loop 201 with a belt inner circumference 202 smaller than the body segment circumference 203 (when muscles are relaxed in the body segment) of the target area of the muscles to be encircled in an initial fitting position,
3) the loop 201 is optionally expanded to a position where the belt inner circumference 202 is larger than the body segment circumference 203 (when muscles are relaxed in the body segment) of the muscles to be encircled in an expanded fitting position,
4) the loop 201 is placed over the target region on the body segment 90 to be restricted in an initial tensioned position where the belt inner circumference 202 shrinks down and compresses some of the tissue in the body segment 90 and becoming substantially co-radial with the body segment circumference 203, thus applying the prescribed pre-tension in the belt and according compression on the body segment,
5) The outer non-stretch layer of the belt 100 is completed by fastening the third fastening means 115 and putting the belt in the open position,
6) The belt 100 is inflated to a closed position,
7) The belt 100 moves from a closed position to a working position and back to the closed position with each contraction/relaxation of the underlying musculature,
8) The belt 100 is removed from the body segment 90.
   As previously stated, some of these steps may be combined or omitted without detracting from the spirit of this invention for a prescription pre-tensioning and positioning system and method.

In addition to the main operation of the belt during a BFR training session, the reader shall also note how the non-linear edging 123 operates to reduce scratching or skin damage to the user. FIG. 2G shows a view of a belt 100 placed around a body segment 90 in an initial tensioned position. Because the belt 100 is trying to shrink smaller than the body segment 90 in order to apply compression, the edges of the belt are effectively trying to cut into the users skin as the skin turns upward and the belt sinks inward. McEwen accurately points out this issue and the importance of edging to reduce a safety hazard to the user. However, the addition of edging adds more parts, labor, and cost to the system and is unnecessary. The applicant's design of a curved, or non-linear edge, shown as a substantially sinusoidal pattern in FIG. 1A provides a protection against skin chaffing but allowing sections of the non-linear edge 123 to fold up as the belt is wrapped circumferentially around the body segment 90. This effect is visible in FIG. 2G. At each location where the portion of the edge juts out as shown by curved semi-circular shapes in FIG. 2G, the belt body 124 will bend because the belt body is trying to close down on the body segment 90 to a belt inner circumference 202 that is smaller than the body segment circumference 203. On the belt body 124 where the inflatable chambers 103 are present, there belt body pushes inward, but the non-linear edge is unable to carry tension and thus cannot restrain the body segment 90. When the non-linear edge 123 bends upward on top of the body segment 90 as shown in FIG. 2G, there is a substantial radius of this bend on the order of at least 1 mm, but often more, and this a smooth soft transition from the non-linear edge to the skin. Experiments were conducted by the applicant wearing belts with and without the non-linear edge 123 and results indicated that the non-linear edge was substantially more comfortable than a linear edge as seen in the prior art.

The applicant also wishes to point out the effects of overlap and wrapping on the occlusion of blood flow. It has been shown in the literature that multiple wraps apply increasingly significant amounts of compressive force to a limb which increases risk of occlusion and underlying nerve damage. Therefore it is the applicant's intention to provide a belt that optimizes the range of muscles to be compressed (to minimize number of belt sizes), but not produce a situation on the smallest end of each belt range where blood flow to the limb could become occluded or the underlying tissue could be exposed to excessively high compressive forces that could damage the musculature. Therefore the applicant suggests limiting the amount of overlap of the inflatable portion of a belt to not wrap the limb more than two full times. The reader shall note that going beyond this limit does not violate the spirit of this invention, it is just a recommended guideline to follow. Overlapping inflatable chambers 103 not only apply additional compressive force but also add in bulk which is counter to the usability of the belt 100 and so it is important to find a balance between the reduction in number of belt sizes and usability of the product. A suitable tradeoff for a belt designed for an 18 cm limb on the small end, may be that the inflatable chambers 103, and corresponding belt body 124, length is 27 cm with a non-inflatable extension of the belt body (for example as shown at the right side of FIG. 1B) to the start of third fastening means 115 of an additional 13.5 cm. Third fastening means may be 5 cm in length by example. So an overall belt 100 length (ignoring the pre-tensioning components like the spring element 114 and second fastening means 111) may be about 45.5 cm (27+13.5+5=45.5 cm). In the case of an 18 cm limb, the inflatable portion of the belt 100 is wrapped 1.5 times around the limb and the non-inflatable extension wraps an additional 18.5 cm to the end of the third fastening means. As pretension was applied already via the applicant's pre-tensioning invention, this additional overlap is laid flat without appreciable additional tensioning need, and therefore the user is not at risk of occlusion as in other “wrap” style devices where each additional wrap is designed to add compression. For such a belt 100, if the spring rate of the spring element 114 were 2 lbs per cm, and minimum designed compression were 2 lbs for a given body segment circumference 203, then the spring length 113 would be 17 cm. Thus the minimal belt inner circumference 202 of the loop 201 is 17 cm and is stretched up to 1 cm to accommodate an 18 cm body segment circumference 203, and therefore applies the specified compression corresponding to approximately 2 lbs of tension in the loop 201 when initially placed on the body segment 90. The reader shall understand that pre-tension in the belt as described in this example and elsewhere in this application is a tensile force that is tangential to the surface body segment 90, but that it corresponds to an inward radial compressive force, which is ultimately the goal of the pre-tensioning system. Therefore, the reader shall understand that the applicant understands the interplay between the dynamics of the tension in the belt itself, tangential to the surface of the body segment 90 and the radially inward compressive force the belt tension produces, which is what compresses the underlying musculature a desired amount. Further, where in the example above a pre-tension force of 2 lb was specified via expansion of the spring element 114 by 1 cm, the reader shall note that when the belt is placed over the limb of the user in the initially tensioned position of FIG. 2C, the belt inner circumference 202 may be larger than the 17 cm because the tissue of the limb will resist compression, but the limb likely will squish somewhat such that the belt inner circumference in the initial tensioned position is not 18 cm. Therefore, the belt tension will not be a full 2 lbs in the initial tensioned position, it will be something less. In this way, the reader shall understand that the relationship between the pre-tension force and limb may be dependent somewhat on the limb composition and therefore the prescription may be related to the body segment circumference 203 at rest, not the final belt tension as observed in the initially tensioned position. The dependency of the final belt tension, and corresponding compressive force on limb composition does not detract from the applicant's invention as the invention always allows a substantially repeatable amount of initial compression to be applied, and applied similarly bilaterally. Further, BMI or other body composition measurements may be taken into account to accurately prescribe an actual belt tension in the initial tensioned position and therefore a known initial compressive force on the limb, as one example.

Finally the applicant wishes to elaborate on the spring element 114 and the ability to adjust or replace this component of the belt 100. The spring element 114 may be modified in length as described previously, or replaced, and in both cases with the goal of changing the spring rate of the loop 201. This purpose may be useful in the cases where the user deviates from the average user either with big strong muscles or thin weaker muscles. In the case of big strong muscles, a higher spring rate may be desired, and in the case of smaller weaker muscles, a lower spring rate may be desired. The ability to modify the spring element, or replace it entirely, gives the added flexibility to the user and makes the part count and SKU count for the manufacturer less, significantly simplifying operations. The reader shall note that the spring element 114 may not be removable or modifiable and yet still cover a large range of users.

If a body interfacing component 301 is provided, and if the body interfacing component is detachable, the steps to get set up shall be substantially similar to those described in the previously filed provisional application referenced herein. Further detail or a possible configuration is described in relation to FIG. 6.

The reader shall also note that while the belt construction described in the preferred embodiment and this operation section is a straight overlap style with multiple inflatable chambers 103, these same principles apply to a single chamber and may be adapted to a fold-back style as well (having a single or multiple chambers). A single chamber configuration is described in the alternate embodiments. A fold-back style as described in the referenced patent applications would simply add a loop coupler (not shown) and have one end of the belt with the third fastening means 115 go through the loop coupler and fold back on itself as in the referenced applications. The applicant has chosen the straight overlap configuration because the pre-tensioning system actually solves the main issue with a straight overlap style belt which is rotation when trying to apply the belt, and therefore can eliminate the loop coupler and complication it adds in manufacturing and bill of materials cost.

The cutouts 122 or otherwise seen as the non-linear edge 123 can also perform a useful function in assisting in the contraction and expansion, and springiness of the belt 100 by reducing the end or edge effects described above. This has been described extensively in the referenced patent applications. The applicant has however, discovered a new benefit to a smooth curving edge design in the reduction of chaffing and improved comfort without the need for additional edging.

The reader shall note that the initial fitting, expanded fitting, initial tensioned, open, closed, and working positions described are for reference comparisons only and, where ranges are given, for example for limb circumferences, the open, closed, and working position may constitute the median value of such range.

### Alternate Embodiment—#1

FIG. 1B shows a modification to the preferred embodiment of FIG. 1A. The main difference in 1B is that the spring element 114 is in communication with the belt body 124 at some point in the mid-section of the belt 100. The method, or options, for attaching the spring element 114 may be substantially similar to the preferred embodiment and materials likewise similar, it is only the location that differs. The optional markings 125 are still present on the inner face 101. A section of the belt body 124 may therefore overhang the spring element 114 and if the spring element is also integrally formed as a fastening means, an optional mating fastening means may be provided on this end of the belt body in addition to the third fastening means 115. As in FIG. 1A, the chambers are depicted as lines for clarity where in reality the inner face 101 is a single flat member comprising the inner belt material 126, and in this case is preferably also the first fastening means 110. Effectively, the belt 100 maybe constructed by welding a piece of integrally formed loop fastener with polyurethane backing servicing simultaneously as the first fastening means 110 and belt inner material 126, to a mating piece of integrally formed loop fastener and PU coating servicing simultaneously as the belt outer material 127 and fourth fastening means 116. The welding die may be formed to create inflatable chambers 103 shown by the lines in FIG. 1B that illustrate the chamber positions and connecting pneumatic pathways, and this welded assembly constituting the belt body 124. The belt body 124 in FIG. 1B may be the entire length of the belt as shown in FIG. 1B with no additional members attached except the spring element 114. Such a construction has been tested and is a minimalistic approach as the belt 100 is formed only of two pieces of integrally formed loop fastener material with a PU backing an a single welding operation. A non-inflatable extension of the belt body 124 can be seen at the right side of FIG. 1B where the belt body has a non-inflatable section separating the inflatable section from the third fastening means 115. This may allow the belt 100 to accommodate a larger range of body segment circumferences 203 applying the applicant's discovery and principle that the entire body segment 90 need not be encompassed by an inflatable section of the belt. While the application has discussed this minimalistic construction concept in relation to FIG. 1B, the reader shall note that such a construction may also extend to any other figures or configurations contemplated by the application, and it is in fact desirably to reduce component count and construction operations for reduction of manufacturing cost and bulk of the final belt 100 when fully assembled.

Operation of this construction is substantially similar to the previous embodiment as the spring element still wraps such that the second fastening means 111 may attach to the first fastening means 110 at some point along the length of the belt 100 to form the loop 201. This embodiment is intended to illustrate the versatility of the design that the placement, location, order of attachment, properties of materials can be varied without departing from the inventive aspects of this specification. The optional portion of third fastening means 115 shown on the left of the figure may exist or may simply be covered up as the right side with third fastening means wraps around the limb to secure the open position.

### Alternate Embodiment—#2

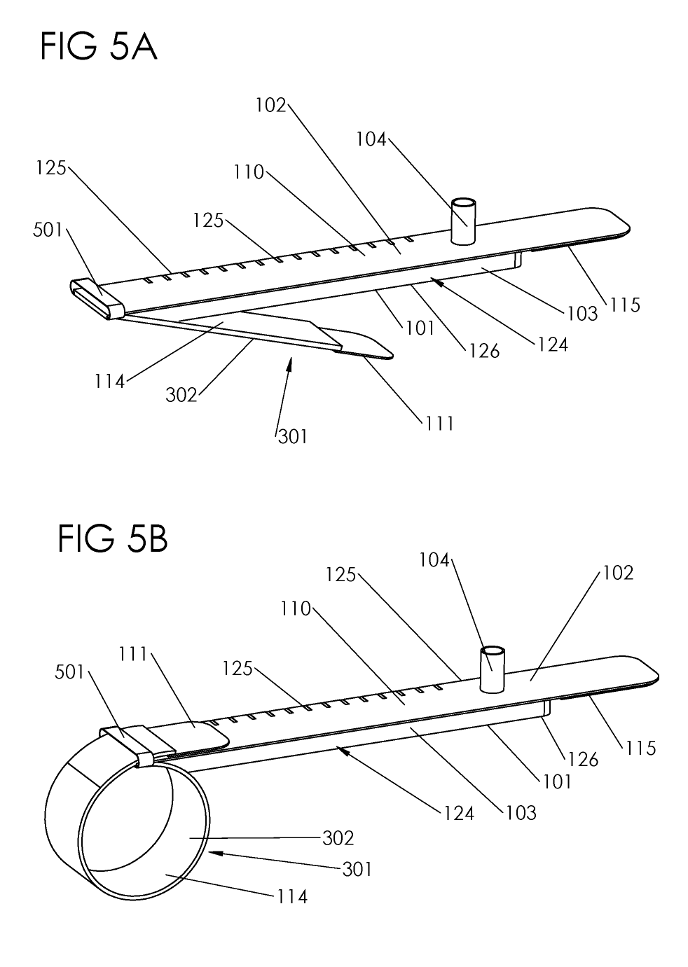

FIGS. 1C, 5A, 5B illustrates yet another configuration of the belt 100 such that the spring element 114 is now facing the same direction as the third fastening means 115. The belt 100 is otherwise substantially similar to the preferred embodiment except that now, optional fourth fastening means 116 is not required and one component or element is removed from the bill of materials to reduce cost. Additionally the first fastening means 110 is now disposed on the outer face 102 instead of the inner face 101 as shown in FIG. 1A. Markings are now placed on the outer face 102 of the belt 100 as shown in FIG. 1C. The location of the spring element 114 may be at one end of the belt body 124, for example the left side of the figure as shown in FIG. 5A,B, or anywhere along the length 112 as shown in FIG. 1C. The lengths of the spring element 114 and location of the spring element may similarly be modified such that the minimum amount of the body segment is covered with inflatable chambers 103 and no more than the maximum amount of overlap of the inflatable chambers is achieved for a given range of muscles to be compressed. Whereas the applicant has suggested an optimal construction is to use integrally formed loop fastener with PU coating for the top and bottom components forming belt body 124, in this configuration only laminated loop fastener is needed as the outer belt material 127, which may further reduce cost.

The operation is again substantially similar to the preferred embodiment in that the spring element 114 and second fastening means 111 are formed into a loop 201, but in this instance the second fastening means is wrapped around clockwise and fastened to the outer face 102 of the belt 100 at the appropriate marking 125 which is also now visible on the outer face of the belt. This looped configuration is illustrated in FIG. 5B. In forming the open position, the third fastening means 115 still overlaps the belt spring 114 in order to create substantially non-stretch outer layer when placed around the body segment 90.

It may be advantageous to lock the spring element 114 to one end of the belt 100, in the case of FIG. 1C the left end, such that when the spring element is wrapped counterclockwise and lies on top of the outer face 102, it does not have a tendency to slip laterally left/right or pull the end of the belt itself counter clockwise. This may be augmented by a lateral stabilizer 501 as shown in FIGS. 5A,B. The lateral stabilizer 501 may be a piece of fabric, plastic, or other suitable material formed into a loop and securing the spring element 114 from sliding left/right. For example, if the second fastening means 111 is positioned midway along the top surface of the outer face 102, the portion of the belt to the left of second fastening means 111 as shown in FIG. 5B will want to be pulled counterclockwise because of tension in the spring element 114. In this manner, it is advantageous to connect the spring element to the end that it overlaps so that tension is retained in the spring element and that end is not allowed to move relative to the spring element. To this effect, the lateral stabilizer 501 shown in FIG. 5A,B may further clamp the spring element to that end. This attachment may be done with any suitable fastening means (not shown) such as a clip, clamp, hook fastener disposed on the outer face 102 in the case the spring element is elastic loop fastener, etc. This additional clamping means may not be necessary if the spring element 114 is positioned at the opposite end, close to the input port or third fastening means, which is the right side as sown in FIG. 1C. In this way the second fastening means 111 is always positioned on the outer face 102 to the left (as shown in FIG. 1C) of where the spring element is attached to the belt body 124. Therefore, all sections of the belt 100 are under tension, whereas if the position of the second fastening means 111 is to the right of the connection point of the spring element 114 and the belt body 124, therefore overlapping the connection point, there is a section of the belt under compression that will tend to move and relax tension on the limb.

### Alternate Embodiment—#3

FIGS. 1D,E illustrate another option for forming the loop where a loop coupler 130 is in communication with the belt spring 114 and provides a means of folding the second fastening means 111 back on itself to connect to either the inner face 101 as shown in Fig D or the outer face 102 as shown in Fig E depending on which direction the second fastening means is looped through the loop coupler. In the case the intention is to fold back onto the inner face 101 as in Fig D, the markings 125 are located on the inner face, and vice versa for the intention of connecting the second fastening means 111 to the outer face 102 in Fig E. The spring element may be attached to one end of the belt 100 or anywhere along the mid-section of the belt as shown in FIGS. 1D,E. In the case the fold-back intention is to the inner face 101, there may also be a fourth fastening means 116 disposed along the outer face 102 as shown in FIG. 1A. FIG. 1E shows an example of how the spring element may itself comprise a fastening means and the length of the spring element may be modified by looping around the loop coupler 130 and attaching back on itself at different positions corresponding to different lengths of the spring element and therefore different amounts of pre-tension.

Operation of this configuration is also substantially similar to the preferred embodiment with the addition that one end of the belt 100 may be looped through the loop coupler 130 and folded back in order to fasten down the second fastening means 111.

### Alternate Embodiment—#4

FIG. 1F depicts a belt 100 with a single inflatable chamber 103, but otherwise substantially similar to the preferred embodiment of FIG. 1A. The purpose again is to illustrate how the inventive concepts are not limited to a particular chamber style or configuration and that the inventive concepts may be adapted to many different configurations, material types, and so forth.

In the case of a single inflatable chamber 103 it may be advantageous to use an additional body interfacing component 301 to space the chamber off the body segment 90 and provide for additional inflation volume as described in previously referenced applications and to counter the effects of kinking when the chamber is turned into a circle. In FIG. 1F, the belt 100 similarly comprises a spring element 114 and second fastening means 111, which may be formed into a loop and attached to the first fastening means 110 which is disposed along a portion of, or all of, the length of the belt. In FIG. 1F, the first fastening means 110 may be disposed across the full width of the belt 100, or only along a portion of the width of the belt. As with all other figures in this application, the reader shall note that the line delineating the single inflatable chamber 103 is only for illustration purposes and does not mean that this is a break in the material or a secondary component. Markings 125 may similarly be disposed along the length of the inner face 101 in order set a predetermined size of loop 201 corresponding to a predetermined pre-tension.

Operation of this configuration is similar to the preferred embodiment and the reader shall note that having a single inflatable chamber vs. multiple inflatable chambers does not appreciably change the operation of the invented pre-tensioning system. In the case of operation of the belt, it is desirable that the materials forming the outer face 102 of the belt are substantially non-stretch such that inflation of the inflatable chamber 103 is directed inward on the body. If the belt 100 is stretchable, then a form of stiffener as described in the prior art may be used to allow the belt to expand without bulging outwards from inflation. In terms of the open position, the open position is defined in the same way as with the preferred embodiment where the belt 100 is not yet inflated but is secured around the body segment 90 with the prescribed pre-tension. During inflation to the desired pressure, the belt inner circumference 202 shrinks radially inward. If the belt was not fabricated with the inner material pre-stretched, kinks may form and a body interfacing component 301 may mitigate against skin pinching or damage. In the case the outer face 102 is substantially non-stretch, there may be slight bulging due to the fabric not being perfectly rigid. The fully inflated position with the fabric compressed and bulged inward on the skin is similarly the closed position as in the preferred embodiment with the exception that the outer wall has not shrunken and if anything, has even expanded slightly from the pressure. The working position during muscle contraction sees the pressure in the belt increase and the belt inner circumference 202 similarly increase, but the outer wall does not appreciably change in circumference, which is in contrast to an elastic belt as in the preferred embodiment where the outer face will increase in length along with the increase in body segment circumference 203.

### Alternate Embodiment—#5

FIG. 1G depicts a belt 100 made of an integrally formed component 400. FIG. 1G depicts the inflatable chamber 103 as a single chamber but the reader shall understand that the chamber design may similarly be multiple chambers as stated earlier in this application and described extensively in the referenced applications.

The integrally formed component 400 may be a molded polyurethane rubber of either varying or uniform thickness, or may be any other suitable material that can be cast or molded to form a bladder and necessary features. Sections with increases thickness may serve to increase the stiffness in certain sections, for example to limit the overall elasticity of the belt body 124. Alternatively, strands of fiber may be embedded during the molding process, or other suitable reinforcement, to similarly reduce the elasticity of certain segments of the belt 100.

Similar to FIG. 1A, one end of the integrally formed component may be elastic and form the spring element 114, or alternatively the spring element may be attached latter via suitable process such as sewing, welding, taping, gluing etc. The spring element 114 may also be formed, or otherwise disposed along a length of the integrally formed component 400, or may face the same direction as the first fastening means 115 as in FIG. 1C. The reader therefore shall note that all configurations and variations that apply to the other figures in this application may be similarly adapted to the concept of an integrally formed component 400, the advantage being that an integrally formed component may provide improvements in manufacture cost, scalability, and overall ease of production.

For illustration purposes, the second fastening means 111 has been depicted in FIG. 1G as a locking means 401, comprised of a series of molded pegs disposed along the length, with matching holes on the end of the spring element 114. This is but one means of creating a locking means 401 and the reader shall note that hook and loop fastener as previously described would also be sufficient if one part (either hook or loop) were disposed along the inner face 101 of the integrally formed component 401.

First fastening means 115 may be disposed on one end opposite the spring element 114 as shown in FIG. 1G, and matching fourth fastening means 116 may be disposed along the outer face 102 of the integrally formed component, as in FIG. 1A. The fastening means may be any suitable means such as hook and loop fastener as shown, or alternatively locking means 401. As previously stated, all other configurations contemplated by the applicant shall be considered adaptable to the integrally formed component, for example in FIG. 1C, the fourth fastening means 116 is not necessary. Similarly to how the applicant provides a locking means 401 instead of first fastening means 110 and second fastening means 111, the third fastening means 115 and fourth fastening means 116 may also be replaced by a locking means 401. Locking means on the outside of the belt 110 may be as previously described in referenced patents such as cam locks, ratchet mechanisms, etc.

An edge 123 may be non-linear as in the preferred embodiment such that it folds up when bent around the body segment circumference 123, and provides a rounded buffer against the user's skin. An advantage to the integrally formed component may be that the non-linear edge 123 may be slightly thickened and rounded such that it further provides a soft transition to the body segment 90.

Operation of the integrally formed component 400 is substantially similar to its counterparts as described in this application, whether that be a single inflatable chamber 103 as in FIG. 1F or plurality of inflatable chambers 103 as in FIG. 1A. The operational steps shall not be repeated here for the sake of brevity, except to say that the selection of the spring factor of the spring element 114, the lengths and relative dimensions of the sections or features of the integrally formed component 400 shall all be such that the inventive concepts around creating a pre-determined loop 201 of appropriate size, and ability to stretch over the body segment circumference 203 shall all be feasible with the integrally formed component.

### Alternate Embodiment—#6

FIG. 1H depicts a belt 100 where a section of the belt body 124 forms the spring element 114. This section may be part of the inflatable chambers 103 or may be non-inflatable, and in the case the section is non-inflatable, inflatable chambers 103 may be disposed on either side of the spring element 114 or only on one side. In the case inflatable chambers 103 are disposed on either side, they may be connected via a tube or other means so as to transmit a flow of gas back and forth, or they may each have separate input ports 104. The reader shall understand that many such combinations and configurations are possible, and breaking the belt 100 up further into more sections than shown in FIG. 1H is similarly possible without departing from the scope and spirit of this invention.

Markings 125 may be disposed along one end of the belt 100 along with first fastening means 110 as shown, and second fastening means 111 disposed at the opposite end. The fastening means may be as described elsewhere in this application, for example hook and loop fastening as preferred in FIG. 1A.

A third fastening means 115, preferably non-stretch, or otherwise high spring constant material, may be disposed along the outer face 102 of the belt 100 and of sufficient length that it can overlap and cover at least the majority, and preferably the full length, of the spring element 114. Other aspects and features of edging and materials, etc. may be similar to other embodiments described in this specification.

Operating the belt 100 of FIG. 1H is again similar to FIG. 1A. The user will form the loop 201 of appropriate belt inner circumference 202 in the initial fitting position to be less than the body segment circumference 203 of the relaxed state of the body segment. This is done by wrapping second fastening means 111 around and overlapping the opposite end of the belt 110, optionally matching with the appropriate, prescribed and pre-determined marking 125 as described in the flow chart of FIG. 4A or 4B. During application to the body segment 90, the portion of the belt body 124 forming the spring element will stretch to cover the body segment 90, and then compress on the body segment in the initial tensioned position. Finally, to complete the pre-tensioning process the third fastening means 115, which is non-stretch or of high spring rate, is placed over the spring element 114 and fastened to first fastening means 110 to lock the outer face 102 in a substantially non-stretch, or high spring rate, configuration. This state now being the equivalent open position. Additional input of air causes similar effects to the preferred embodiment and the rest of the operation need not be repeated here, as it is substantially similar to the preferred and other embodiments. In this manner the belt 100 is initially allowed to stretch over the body segment 90 and relax on the segment in the initial tensioned position and then it is locked in this position with the a portion of non-stretch, or substantially non-stretch material to secure the open position.

### Alternate Embodiment—#7

FIGS. 3, 6 depicts a belt 100 with an optional and separable body interfacing component 301. The body interfacing component, its benefits, properties, and uses has been extensively covered in the referenced applications and all such characteristics and associations shall be considered relevant as well to this invention.

The body interfacing component 301 may be fixed permanently to the belt 100 via suitable means such as sewing, bonding, welding, etc, or may be detachable via suitable means such as hook and loop fastener, static friction, adhesive, etc. as shown in FIG. 6. Alternatively the body interfacing component 301 may simply be loose and not connectable to the belt 100.

The body interfacing component 301, in addition to other properties discussed previously, may absorb sweat and act as a sanitation barrier between the belt 100 and the user to avoid the transfer or bacteria or other microbes such that the belt itself does not need to be washed, only the body interfacing component does. The body interfacing component 301 in this way can also be disposable, in which case it is preferred the material also be bio-degradable.

The applicant describes throughout this application how various components may be combined to eliminate parts and reduce cost and one combination is for the body interfacing component to be made of elastic material, for example neoprene with a thickness between 1 mm and 3 mm, and used as the spring element 114. This effectively is just changing the preferred material of the spring element form an elastic webbing to neoprene and simultaneously providing the benefits of the body interfacing component and the pre-tensioning capabilities of the spring element. As shown in FIG. 6, the belt may not comprise a separate spring element 114 in this case.

The body interfacing component 301 may be the thickness as suggested in the referenced applications or may be thinner and still serve as an adequate sanitation barrier. The body interfacing component may comprise an optional frictional surface 302 to help resist rotation and hold position on the body segment 90. Alternatively the body interfacing component 301 may not comprise the frictional surface 302 in which case the body interfacing component may be free to adjustably slide up and down the body segment 90 or rotate around the body segment. The body interfacing component 301 may be adjustable in circumference and may serve to provide the initial compression on the limb with a fastening means to then attach the belt 100 which may then wrap around the limb and secure the open position as shown in the configuration of FIG. 6.

Operationally the body interfacing component 301 may be placed over the body segment 90 prior to the application of the belt 100, or the body interfacing component may first be placed on the belt and the assembly stretched and placed over the body segment. How the configuration on the body segment 90 is achieved is not critical as long as in operation the body interfacing component 301 is placed between the belt 100 and the body segment 90.

In the case the body interfacing component 301 is separate from the belt 100, for example as in FIG. 6, the body interfacing component may be configured to the initial fitting position to apply a certain amount of pretension to the limb by adjustment of its circumference prior to placement on the limb or during placement on the limb as described in FIGS. 4A,B. As described this may optionally be prescribed by markings as seen in FIG. 6, or simply by feel. The body interfacing component may then be stretched (in the case of the procedure of 4A) to the expanded fitting position for placement over the limb. The body interfacing component 301 may then be placed on the limb while applying a desired amount of pre-inflation compression, therefore constituting the initial tensioned position as elsewhere described in this application. The belt 100 may then optionally be anchored to the body interfacing component 301 via suitable means, such as a button snap or hook and loop fastener or hook, etc. and then wrapped around the limb and fixed to itself to for a non-stretch outer layer comprising the open position. In this manner the reader shall understand the applicant's invention as a broad concept, and that the various states described: initial fitting position, expanded fitting position, and initial tensioned position, open position, etc. may be achieved by separate components independently, which may then be combined to arrive at the same end result.

### DESCRIPTION—CONCLUSION, RAMIFICATIONS, SCOPE

Thus the reader will see that the various inventions described herein provide an economical way to easily create a multifunctional, safe, inexpensive, easy to use blood flow restriction system and belt for incorporation therein, with an additional feature of a simple to use, prescriptive, and repeatable pre-tensioning system. Additionally the reader will see that inventions described herein may take advantage of current mass production processes to keep the additional cost minimal, and that by reducing component count, the applicant has not only reduced the manufacturing costs but reduce the level of complexity of operating the system, and reduced the bulk of the system which, since it is a wearable product to be used during exercise, is a significant factor as Sato himself describes. The reader shall further note that the applicant has invented a way to solve one of the biggest complaints with prior art devices, including the applicant's where the pre-tensioning of the belt is a critical aspect to the effectiveness, and therefore needs to be quantified, and made easily reproducible.

While the above description contains specificities, these should not be construed as limitations on the scope of the invention, but rather as an exemplification of preferred embodiments thereof. Many other variations are possible as described below.

### Elasticity

Where the term elasticity is used in this specification, the reader shall note that the applicant may be referring to an elastic property of the construction, and not necessarily that the materials themselves, as individuals or when combined, have elastic properties, i.e. form a material or combinations of materials that themselves stretch in nature. The applicant acknowledges that the materials may have elastic properties themselves, but this may only be an option, not a requirement.

Similarly, the reader shall note that the applicant, when defining materials as non-stretch, or inelastic, understands that all materials stretch to some degree when a force is applied. The applicant's description of the term ‘inelastic’ or ‘non-stretch’ within the context of this application shall therefore be construed to comply with the applicant's intent and purpose for each such element within each embodiment as described. For example, a 200 denier ballistic nylon fabric, coated with polyurethane may stretch less than 1% when subject to a stretching force, but such material may be considered inelastic, or otherwise known as ‘non-stretch’ in the industry, and considered non-stretch in the context of this invention when compared to prior art latex rubber bladders, which may stretch by a substantially greater amount, for example perhaps even 100% for the same given applied force and material dimensions. The reader shall therefore refer to the intended use, desired traits, and function in the herein described invention when taking into context a material property being considered non-stretch vs. high spring rate vs. elastic.

Materials described similarly may be understood to encompass combinations of materials, varying material properties such as durometer or elastic modulus, lengths and widths, and profiles, which affect properties such as elasticity and coefficient of friction, and all such combinations may be considered within the scope of this invention. Further the reader shall note that where a material may be discussed as elastic, a non-elastic material may be combined with an elastic material to form what would be considered the original member (or visa-versa), but which is now two components and may not specifically match the description herein. However, in such cases, the reader shall note that the applicant has in fact considered that materials may be combined to perform the function of the elements of the inventions described herein, but has not made all such descriptions because of the endless possible combinations possible. All such combinations yield the same result as originally disclosed that the spring element member has some degree of elasticity. Yet another example is the reader may note that some element properties may be altered to remove various components. For example the belt may have some degree of elasticity in order to compensate for muscle contraction. Again, the reader may note that all such combinations or omissions of components, or altering of various component properties may be considered within the scope of this invention.

The reader shall also note that the pre-tension concepts may not necessarily require the use of elastic materials. The spring element 114 itself, while desired to be of a low spring constant, may be non-stretch or of a significantly high spring constant. If fact the spring element could be removed altogether and replaced by an extension of the inner belt material 126, or outer belt material 127. Because of the compressibility of the limb, and the procedure of 4B, where the initial tensioned position may be created while simultaneously applying the belt 100 to the body segment 90, it is possible for a user to apply the belt to the limb without an elastic element in the belt. The result is a relatively tight belt that doesn't stretch on the limb that may be less comfortable, but nonetheless serves the purposes of forming an initial tensioned position. The belt 100 may then be positioned or rotated and prepared to lock the belt position down to the open position by securing the third fastening means 115. In FIG. 1A for example the spring element 114 could be removed and the second fastening means 111 attached to the end of the belt body 124. In applying the belt, the user could take the end with the third fastening means 115 and wrap this belt clockwise around the limb and securing the second fastening means 111 to an appropriate location on the first fastening means 110, leaving a “tail” with the third fastening means. There would be substantially little elasticity in this initial tensioned position. The belt could be rotated or adjusted on the limb and then the “tail” pulled tighter if desired and placed down on the fourth fastening means 116 to lock the outer circumference in the open position. Therefore the reader shall understand that the desired elastic aspects may be altered or removed to further simplify the construction as long as the general idea of providing an initial tensioned position first is followed by locking the belt into an open position. The reader shall finally note that this configuration described also solves the deficiency noted earlier with Sato's and prior art overlap style belts that the overlapped section can slip out easily to one side. As just described, but securing second fastening means 111 to the underside of the belt body 124, the applicant has prevented this end with second fastening means from moving laterally relative to the belt body and therefore it will not slip out to the side and lose compression.

### Belt Shapes/Sizes

For example, in the case of belt shape, the inflatable portion of the belt may be of any suitable geometry, size and shape to provide sufficient blood flow restriction as discussed above. Belts may come in multiple lengths and widths to accommodate a range of individuals, and not necessarily minimized in the number of variations, but rather targeted toward a specific range of limb girths, or user types. It may be noted that wider cuffs have been shown to restrict flow to the same extent as narrower cuffs, but at lower pressures and may offer more comfort for certain applications that don't require dynamic movements. Such width variations for a specific user, such as assisting the elderly, may improve comfort while maintaining effectiveness. Belt shapes which employ enough tissue displacement to restrict venous return, such as some examples described herein, may be used, and may not necessarily cover the entire limb. All such configurations of profiles, sizes of belts, gas bladders, locations placements of such belts on the body, and bladders on belts, may be considered within the scope of this application.

Discussed previously in the application are guidelines around how to expand the usable range of limb girths for a particular belt by modifying the inflatable portion and the non-inflatable portion. The reader shall note that the belt may be segmented in to multiple inflatable or non-inflatable portions and as long as the principles around targeted inflation as described in previously applications as been sufficient to restrict an adequate flow of blood for effectiveness, any and all variations in belt construction, lengths of inflatable portions, and lengths of non-inflatable portions shall be considered within the scope of this invention. In general as long as enough of the body segment is covered by inflatable portions to restrict flow, the non-inflatable portion may be continually increased until this no longer holds true, thereby increasing the usable range of the belt. In staying with the principles of the applicant's invention, lengthening members may also be applied to the belt as an effective “extension” for the belt. For example, if a belt is desired to be lengthened to accommodate a larger range of limb sizes, a mating fastening means may be attached to third fastening means 115. This mating fastening means may be coupled to an extension member of some length, and then a replacement fastening means coupled on the opposite end, the replacement fastening means being similar to the third fastening means. This simple addition can be permanent or removable and shall be understood to effectively lengthen the belt 100.

As previously described, the quantity, size and shape of the inflatable chambers may similarly be modified to improve comfort, reduce edge effects, and improve the shrinking effectiveness of the belt as described herein and in referenced applications. While a majority of the figures in this application depict a multi-chamber belt, the reader shall understand that in all cases, a single chamber belt will also function.

With regards to minimal coverage and maximum overlap, the reader shall understand that the applicant's suggestions herein are guidelines and not rules that limit the scope of this invention or possible configurations. As the width of the belt can be varied to reduce the effect of a given pressure, this would then allow for more overlap before reaching the same level of occlusion risk. It may be advantageous to reduce the belt width and increase the overlap in order to reduce the amount of belts needed to cover all practical limb sizes from, for example, 18 cm up to 100 cm. The applicant has provided basic concepts and formulations herein for calculating and making design decisions based on certain minimum coverage/maximum overlap combinations and this is what is important, not what the specific numbers are described by the applicant for those design guidelines.

It may be advantageous to provide a printable surface or section of the belt used for branding or labeling purposes. To this end, the portion of the belt body 124 of FIG. 1A (or corresponding section in other embodiments) is meant to overlap the opposite end may be extended or otherwise intended not to be overlapped itself. This section of the belt body 124 that will never be overlapped does not therefore required application of fourth fastening means 116 on this section. This section can be seen more clearly in FIG. 1B as the printable section 131 designated by dashed lines. This section may reside to one side of the input port 104 for example, but could also extend past the input port towards the section to be overlapped. The printable section 131 may be inflatable or non-inflatable. In one configuration, the printable section 131 may be formed by attaching a piece of printable fabric, for example loop fastener with the loop side against the body segment 90 and the printable backing exposed to the outside world. In this manner a substantial area is exposed for printing customized branding, warnings, serial numbers or other visual items. As this piece may be separate from the rest of the construction, it may be made from different colors and fabricated separately such that many different styles and options are available in a cost effective manner where the expensive components of the construction are all the same, and simply attached to whichever specific printable section 131 is desired for that manufacturing lot.

### Open, Closed, Working Positions

The reader shall further note that the open, closed, and working positions as defined above could also constitute the small or large end of any range or spectrum described in this specification. For example, if a belt size range is targeted at limbs of 18 cm-33 cm, the open position could be a closed circumference of any size that fits over a limb up to at least a 33 cm limb. In case the specified limb is 18 cm for example, the open position may be slightly smaller than 18 cm because of pre-tension has compressed the underlying limb slightly, thereby reducing its circumference. Additionally in the case the pretension prescription is substantially zero initial compression, as may be the case of an elderly person using the belt, the open position may itself be co-radial with the body segment circumference 203. The reader shall note that it is possible to operate the invention without any pretention and therefore an inner belt circumference 202 that is larger than the body segment circumference 203 is formed, and that this configuration and method are covered in previously filed applications by the applicant. Similarly the closed position in this case is less than the open position by some amount when referring to the inner circumference of the belt bladder. The reader shall note that in the case of a single bladder, the outer circumference of the belt does not shrink, and may even slightly expand under pressure when referring to the open, closed, and working positions in this specification the application is referring to the inner face 102 of the bladder or otherwise termed the belt inner circumference. The specific amount of shrinkage of the belt inner circumference depends on a variety of factors such as the starting open position and tension on the limb, the amount of blood flow restriction desired, the amount of adipose (fat, muscle, bone) or otherwise the makeup of the body segment 90, and the amount of pressure applied to reach the desired restriction. Similarly, the working position when wrapped around a limb entails a belt inner circumference that is greater than the closed position belt inner circumference, and less than, or up to the nominal open position inner circumference, thereby illustrating that the belt inner circumference shrinks when moving from the open position to the closed position, and expands when moving from the closed position to the working position. In the case of the barrel inflatable belt design the nominal belt circumference similarly shrinks and expands as previously described in the reference applications.

As in the example above the open position may be slightly less than 18 cm in circumference, the closed position may be 16 cm in circumference, and maximum working position may be 17 cm in circumference. Or, if more compression is desired the open position may be 18 cm in circumference, the closed position 15 cm in circumference and the working position 17 cm in circumference. Therein, the reader shall understand that these terms may vary considerably depending on a specific situation and the specification and appended claims shall take into account all possible scenarios and interpreted to the broadest extent.

### Combinations of Materials and Design Elements

The reader shall note that many design elements and material property combinations have been discussed and that these factors: number of inflatable chambers 103, height to width ratio of the chambers, width of the belt 100, range of limb circumferences to cover, belt material properties, cutouts 122, body interfacing component 301, and targeted compression vs full encirclement of the limb, to name a few may all be combined in full or in part, altered in some way, shape, quantity or form, or otherwise modified so as to improve or alter the properties of the belt. For example, there may be as few as 1 chamber and this chamber may be biased inward against the limb, even though it may not be as effective as having more chambers. Similarly, there may be 50 chambers for full encirclement of a large limb, and still achieve some amount of shrinkage and provide elasticity.

Materials and components may be combined to reduce the number of parts and operations needed to fabricate the belt 100 and the applicant has discussed previously for example how inner belt material 126 and first fastening means 110 may be a single piece of loop fastener coated with PU, and likewise the outer belt material 127 and fourth fastening means 110 may be combined. The reader shall understand that such combinations and advancements in material selections are known to those skilled in the art and shall be considered within the scope herein. Similarly the spring element 114 may itself be elastic loop fastener thereby forming a combination of one of the fastening means and the spring element into one component vs. two as this design element was described previously.

The applicant has covered in this application, the physics, mechanical properties, and tradeoffs of these various important properties and design elements, and the reader shall understand that all such combinations and modifications of these features that affect or improve the properties and function of the belt 100 for restricting blood flow in a limb, shall be considered within the scope of this invention, and the applicant's invention shall not be limited solely to the combinations depicted in the figures or described in this specification, but rather by the broad concepts discussed herein.

### Belt Materials

Various belt and blood flow restriction system designs have been described herein and in the referenced applications, and various material constructions and configurations have likewise been disclosed. Various components being elastic, and relative degrees of elasticity have further been noted. The reader may note that for the sake of brevity, not all such combinations and material types have been discussed, but all such combinations, material properties or configurations may be considered within the scope of this invention. For example, in the case of the fastening means: cam-locks, ratchets, and hook and loop fasteners have been described or referenced, however many other such means of fastening two objects together may be used such as a high friction joint tri-glide style mechanism, glues or adhesives, ropes or knots, mechanical hooks, buttons, racks and pinions, high friction surfaces, etc may be consider encompassed within the term fastening means and this term interpreted as broadly as possible. Further, in the case of elastic members or fabrics, polyurethane coated fabrics may be substituted for PVC coated fabrics or a similar material, and urethane molds, but may be of latex rubber, or similar material. In all such cases where specific materials are called out, the readers may understand that, this specification is but one example, and as long as the general concept described is achieved, the specific material, or specific property thereof, is not a requirement of the invention.

### User

The user in the context of this application may be deemed to mean the person using the inventions described. This may be a client, patient, instructor, personal user, doctor, athletic trainer, coach, etc.

### General

One skilled in the art will recognize any minor modifications that would be needed for such an intermingling and such modifications may be considered within the scope of this specification and claims. Further, it may be recognized that many of the components described may be combined into a single object via different manufacturing processes such as welding, injection molding, casting, etc. While the applicant discusses some of these options briefly in the application, it may be recognized any and all combinations of the components discussed herein may be considered within the scope of this application and covered by the claims written. Similarly, it may be recognized that many components in the system and their connection points, or connection means, may also be interchanged or rearranged to achieve the same effect as the disclosed configurations.

In general, valves, and valve types, fastening means, such as cam locks, hook and loop fasteners, ratchet mechanisms, spring elements, inner and outer belt materials etc. may be interchanged, used in quantities of more than one, altered in width, length, or profile, employed in conjunction of overlapping belt styles, or doubling back of belt styles for locking, or more complicated belt designs such as those shown in patents to Sato, and the inventions disclosed herein may be considered to have encompassed all such permutations and combinations of such components. Yet another example is the belt may have two input ports, one to allow air in and another in communication with an outlet system such as a pressure relief valve. While such design is not shown in the figures above, the reader may note this concept is another example of how multiple items may be employed, and components shifted within the system to connect with different components, while the same overall system and effectiveness is maintained. Further still, the location and placement of various elements may be moved and altered such that they appear to differ from the figures shown, and description attached, however, all such configurations and combinations may be considered within the scope of the inventions disclosed herein. For example, in the case of the hook and loop fastener shown on the belt in FIG. 1A, the hook and loop fastener may be exchanged and the function still maintained. In addition, the location of the input port may be in the middle of the belt instead of on one end. The body interfacing component 301, such as neoprene rubber, shown in FIG. 3 may be permanently attached to the inflatable bladder, or it may be removable as in FIG. 6. If removable, the attachment means may be for example, hook and loop fasteners, and the fasteners may be along the edges or may run along the full width of both the inflatable bladder and body interface component. In the case the hook and loop fasteners run along the full width, they may be elastic such that the inflatable bladder may still inflate against the user's limb. As illustrated, there are many constructional permutations and combinations, and options for various material properties that yield satisfactory results in a belt for use in a blood flow restriction system, and all such combinations and permutations and material property choices may be considered within the scope of this invention.

### Belt Configurations

As has been discussed in both this application and patents to Sato, there are a variety of ways to form a belt around a user's limb and each has some advantages and disadvantages as discussed in the various applications. The reader may recognize that the inventive concepts disclosed herein may be considered adaptable, by changing, but not limited to, the following: size, length, location, neighboring components, adding or removing one or more components, such as a loop coupler material properties, such as elasticity, etc. Such modifications represent numerous permutations and configurations which are too many to reasonably depict and describe herein, however the reader may understand that the applicant has thought of such reasonable modifications, and as such may be considered part of the scope of this disclosed invention. Additionally the bladder component may be detachable from the rest of the belt and may be replaceable with a bladder of different length in order to accommodate different limb sizes and the belt may be adaptable. The reader shall recognize that the applicant contemplates such configurations and when a belt 100 construction is completed at some point during the usable life cycle of the belt, from the point it is manufactured to the point it is thrown away, and this construction has the functionality and inventive concepts disclosed herein, such constructions or configurations shall be considered within this scope. In such a manner the pre-tension system may be considered completely separate from the inflatable portion where the pre-tension components are not themselves inflatable, but this non-inflatable pre-tension system may wrap over or otherwise be combined with an inflatable bladder, for example via hook and loop fastener, to form a belt substantially similar in construction to what is disclosed herein, for example as depicted by the body interfacing component 301 in FIG. 6.

### Purpose of Belts

The previous discussion has extensively covered the use of the applicant's invention and belt 100 design in the context of a muscle development tool used for BFR training. However the applicant would like to point out that the generic construction can be useful for wrapping anything tight against the body, and not necessarily for the purpose of restricting blood flow.

Some purposes for this could be affixing, or otherwise integrating the design to clothing to pull a section of clothing tight against the arm, leg, or even waist as in a traditional belt. Such consideration may be useful for example in conjunction with an unweighting system where it is often difficult to have garments grab, or adhere to the body as a vertical force is applied. In this circumstance, the applications invention may serve to aid in wrapping or grabbing onto a body in order to lock and provide an anchor off of which to pull. The belt could be applied external to the garment or integrated into the garment, for example a pair of shorts, or a shirt. The shrinking belt portion may warp around the chest, or waist, or arms, or any part of the body so as to fulfill its purpose. Any sort of lifting or force transfer apparatus may be connected to the belt itself, or to a structure that is connected to the belt, such that the load is eventually transferred to the body in such a way that the belt helps with efficient and comfortable load transfer.

Another example may be applying compression in the case of a wrapping an ice bag or heating pad to the limb. In these cases, it is often difficult to get a good wrap on the limb, or requires a lot of plastic to wrap around in order to stay in place when the person stands up or wants to move from one spot to another. In these cases, a fast inflating sleeve that surrounds an ice bag or heating pad, and that secures it tight to a limb may be particularly useful for quick on/off, and without wasting materials such as plastic wrap that is commonly used.

In any of these, or related use cases, the reader shall understand that all the designs, aspects, characteristics, methods, and inventions described in this application shall be applicable to such use cases, and this generic concept of securing a belt to a body segment make take advantage of the inventions described in this specification and the provided claims.

The reader shall note that these are but a few examples, and the general concept of using the applicants' invention to secure the belt to the body, or use the belt to secure an object to the body shall be considered within the scope of this invention.

Although the description above contains many specifications, these should not be construed as limiting the scope of the invention but as merely providing illustrations of some of the presently preferred embodiments of this invention. Thus the scope of this invention should be determined by the appended claims and their legal equivalents, rather than by the examples given.

I claim:

1) A blood flow restriction device for securing to a body segment comprising:
a belt comprising an inner face and an outer face, the belt further having a belt length and forming a loop which defines a belt inner circumference and an initial tensioned position when the loop is positioned around a body segment; and
at least one inflatable chamber coupled to the belt.

2) The blood flow restriction device of claim 1, further comprising:
a first fastening means disposed along at least a portion of the belt length; and
a second fastening means which is releasably attachable to the first fastening means, the second fastening means being adjustable relative to the first fastening means to adjust the belt inner circumference when released and set the belt inner circumference when attached to the first fastening means.

3) The blood flow restriction device of claim 2, further comprising:
the first fastening means being attachable to the second fastening means to set the belt inner circumference in an initial fitting position before the belt is positioned around the body segment.

4) (canceled)

5) The blood flow restriction device of claim 2 wherein;
the belt inner circumference in the initial fitting position is smaller than a body segment circumference of the body segment where the belt is to be located, the belt inner circumference being selected based upon the body segment circumference prior to fitting the belt around the body segment.

6) (canceled)

7) (canceled)

8) (canceled)

9) (canceled)

10) (canceled)

11) (canceled)

12) (canceled)

13) The blood flow restriction device of claim 2, further comprising at least one spring element, wherein at least one spring element of the belt has an elastic property.

14) (canceled)

15) (canceled)

16) (canceled)

17) The blood flow restriction device of claim 13 wherein the spring element is in communication with an end of the belt, the end being manipulate-able by the user to alter the inner circumference when the first fastening means is detached from the second fastening means.

18) (canceled)

19) (canceled)

20) (canceled)

21) (canceled)

22) (canceled)

23) (canceled)

24) (canceled)

25) (canceled)

26) The blood flow restriction device of claim 1 wherein the belt inner circumference formed by the loop is less than the belt length, and a portion of the belt not forming the loop is manipulated by the user to secure a third fastening means.

27) (canceled)

28) (canceled)

29) (canceled)

30) (canceled)

31) (canceled)

32) (canceled)

33) (canceled)

34) The blood flow restriction device of claim 13, the belt inner circumference of the loop being expandable to an expanded fitting position when the belt is manipulated into the initial tensioned position, the belt inner circumference being able to temporarily increase for placement over a target section on the body segment without detaching the second fastening means from the first fastening means.

35) (canceled)

36) (canceled)

37) (canceled)

38) (canceled)

39) (canceled)

40) (canceled)

41) (canceled)

42) The blood flow restriction device of claim 1 wherein the belt is locatable around the body segment in the initial tensioned position and the loop has a first stretch factor in the initial tensioned position.

43) (canceled)

44) (canceled)

45) (canceled)

46) (canceled)

47) (canceled)

48) (canceled)

49) The blood flow restriction device of claim 42 wherein, the belt further comprises a third fastening means being connectable along a length of the belt to maintain the belt inner circumference in an open position and the belt has a second stretch factor in the open position.

50) (canceled)

51) (canceled)

52) (canceled)

53) (canceled)

54) (canceled)

55) (canceled)

56) The blood flow restriction device of claim 49 wherein the third fastening means communicates with a fourth fastening means such that the third fastening means maintains the belt inner circumference.

57) (canceled)

58) (canceled)

59) (canceled)

60) (canceled)

61) (canceled)

62) (canceled)

63) The blood flow restriction device of claim 49 wherein the at least one inflatable chamber is inflatable to move the belt from the open position to a closed position.

64) (canceled)

65) The blood flow restriction device of claim 63 wherein the closed position of the belt correlates to a compressive force on the body segment sufficient to restrict a flow of blood in the body segment by a desired amount.

66) (canceled)

67) (canceled)

68) The blood flow restriction device of claim 63, wherein:
the belt is movable from the closed position to a working position, the working position being a position in which the body segment expands within the loop due to contraction of musculature constrained by the loop when in use.

69) (canceled)

70) (canceled)

71) (canceled)

72) (canceled)

73) (canceled)

74) (canceled)

75) (canceled)

76) (canceled)

77) (canceled)

78) (canceled)

79) (canceled)

80) (canceled)

81) (canceled)

82) (canceled)

83) (canceled)

84) (canceled)

85) (canceled)

86) (canceled)

87) (canceled)

88) (canceled)

89) (canceled)

90) (canceled)

91) (canceled)

92) (canceled)

93) (canceled)

94) (canceled)

95) (canceled)

96) (canceled)

97) (canceled)

98) (canceled)

99) (canceled)

100) (canceled)

101) The blood flow restriction device of claim 1, wherein:
the belt further comprises a body interfacing component.

102) (canceled)

103) (canceled)

104) (canceled)

105) (canceled)

106) (canceled)

107) (canceled)

108) (canceled)

109) (canceled)

110) (canceled)

111) (canceled)

112) (canceled)

113) (canceled)

114) (canceled)

115) (canceled)

116) (canceled)

117) (canceled)

118) (canceled)

119) (canceled)

120) (canceled)

121) (canceled)

122) (canceled)

123) (canceled)

124) (canceled)

125) (canceled)

126) (canceled)

127) (canceled)

128) (canceled)

129) (canceled)

130) (canceled)

131) A method for securing a blood flow restriction device to a body segment comprising:
providing a blood flow restriction device having a belt comprising an inner face and an outer face, and at least one inflatable chamber, the belt forming a loop with a belt inner circumference in an initial tensioned position when positioned around the body segment before inflating the inflatable element; and
positioning the body segment in the loop.

132) The method of claim 131 further comprising the step of: providing a first fastening means and second fastening means in communication with the belt and coupling a first fastening means to a second fastening means to form the loop.

133) The method of claim 132 wherein the coupling is done prior to placement on the body segment to form an initial fitting position.

134) The method of claim 131 further comprising the step of: coupling a first fastening means to a second fastening means to define an initial tension position.

135) (canceled)

136) (canceled)

137) (canceled)

138) (canceled)

139) (canceled)

140) (canceled)

141) (canceled)

142) (canceled)

143) (canceled)

144) (canceled)

145) (canceled)

146) (canceled)

147) (canceled)

148) (canceled)

149) (canceled)

150) (canceled)

151) (canceled)

152) (canceled)

153) (canceled)

154) (canceled)

155) (canceled)

156) (canceled)

157) (canceled)

158) (canceled)

159) (canceled)

160) (canceled)

161) (canceled)

162) (canceled)

163) (canceled)

164) (canceled)

165) (canceled)

166) (canceled)

167) (canceled)

168) (canceled)

169) (canceled)

170) The method of claim 133, further comprising forming the loop prior to applying to the body segment and temporarily stretching the loop to an expanded fitting position for placement over a target section on the body segment without detaching the first fastening means from the second fastening means.

171) (canceled)

172) (canceled)

173) (canceled)

174) (canceled)

175) (canceled)

176) (canceled)

177) (canceled)

178) (canceled)

179) (canceled)

180) (canceled)

181) (canceled)

182) (canceled)

183) (canceled)

184) (canceled)

185) The method of 131 further comprising releasing the loop on the body segment, the loop being sufficiently elastic to reduce in circumference to create enough frictional force against the body segment to secure the loop to a location and orientation on the body segment, the frictional force being sufficient weak as to allow manual adjustment, and further adjusting one of a rotational orientation or a longitudinal position of the belt on the body segment.

186) (canceled)

187) (canceled)

188) (canceled)

189) (canceled)

190) (canceled)

191) (canceled)

192) (canceled)

193) (canceled)

194) (canceled)

195) (canceled)

196) (canceled)

197) (canceled)

198) (canceled)

199) (canceled)

200) (canceled)

201) (canceled)

202) (canceled)

203) (canceled)

204) (canceled)

205) (canceled)

206) (canceled)

207) (canceled)

208) (canceled)

209) (canceled)

210) (canceled)

211) (canceled)

212) (canceled)

213) (canceled)

214) (canceled)

215) (canceled)

216) (canceled)

217) (canceled)

218) (canceled)

219) (canceled)

220) (canceled)

221) (canceled)

222) (canceled)

223) (canceled)

224) (canceled)

225) (canceled)

226) (canceled)

227) (canceled)

228) (canceled)

229) (canceled)

230) (canceled)

231) (canceled)

232) (canceled)

233) (canceled)

234) (canceled)

235) (canceled)

236) (canceled)

237) (canceled)

238) (canceled)

239) (canceled)

240) (canceled)

241) (canceled)

242) (canceled)

243) (canceled)

244) A method for securing to a body segment comprising:
providing a body interfacing component, securing the body interfacing component to a body segment in an initial tensioned position,
securing an inflatable belt to the body interfacing component with an attachment means,
wrapping the belt around the body interfacing component and securing the belt in an open position.

245) (canceled)

246) (canceled)

247) (canceled)

248) (canceled)

249) (canceled)

250) (canceled)

251) (canceled)

252) (canceled)

253) (canceled)

254) (canceled)

255) (canceled)

256) (canceled)

257) (canceled)

258) (canceled)

259) (canceled)

260) (canceled)

261) (canceled)

262) (canceled)

263) (canceled)

264) (canceled)

265) (canceled)

266) (canceled)
